(19) United States
(12) Patent Application Publication
(10) Pub. No.: US 2007/0167901 A1
(43) Pub. Date: Jul. 19, 2007

(54) Self-sealing residual compressive stress graft for dialysis

(76) Inventors: Judson A. Herrig, Elko, MN (US); Robert J. Ziebol, Blaine, MN (US); Christopher H. Porter, Woodinville, WA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/600,589

(22) Filed: Nov. 16, 2006

Related U.S. Application Data
(60) Provisional application No. 60/737,658, filed on Nov. 17, 2005.
(60) Provisional application No. 60/763,240, filed on Jan. 30, 2006.

(51) Int. Cl. A61M 39/02; A61F 2/06

(52) U.S. Cl. 604/6.16; 623/1.35; 623/1.44

(57) ABSTRACT

Vascular access systems for performing hemodialysis are disclosed. Some embodiments relate to vascular access grafts comprising an instant access or self-sealing material reinforced with expanded PTFE to resist stretching of the instant access material and thereby resist leakage associated with stretching or bending. The graft may comprise two end segments comprising ePTFE without the instant access material to allow easier anastomosis of the graft to veins and arteries. The graft may have a unibody design or have modular components that may be joined together to create a graft with customized length or other features. One or more sections of the graft may also be cut or trimmed to a custom length.

### CROSS-REFERENCE TO RELATED APPLICATIONS

The present application claims priority under 35 U.S.C. §119(e) to U.S. Provisional Application Ser. No. 60/737,658 filed on Nov. 17, 2005, and to U.S. Provisional Application Ser. No. 60/763,240 filed on Jan. 30, 2006, incorporated herein by reference in their entirety. The present application also incorporates by reference in their entirety all of the following applications: U.S. application Ser. No. 11/216,536 filed on Aug. 31, 2005, which is a continuation-in-part of U.S. application Ser. No. 10/962,200 filed on Oct. 8, 2004, which claims priority under 35 U.S.C. §119(e) to U.S. Provisional Application No. 60/509,428 filed on Oct. 8, 2003, and to U.S. Provisional Application No. 60/605,681 filed on Aug. 31, 2004.

### BACKGROUND OF THE INVENTION

In the United States, approximately 400,000 people have end-stage renal disease requiring chronic hemodialysis. Permanent vascular access sites for performing hemodialysis may be formed by creating an arteriovenous (AV) anastomosis whereby a vein is attached to an artery to form a high-flow shunt or fistula. A vein may be directly attached to an artery, but it may take 6 to 8 weeks before the venous section of the fistula has sufficiently matured to provide adequate blood flow for use with hemodialysis. Moreover, a direct anastomosis may not be feasible in all patients due to anatomical considerations. Other patients may require the use of artificial graft material to provide an access site between the arterial and venous vascular systems. Although many materials that have been used to create prosthetic grafts for arterial replacement have also been tried for dialysis access, expanded polytetrafluoroethylene (ePTFE) is the preferred material. The reasons for this include its ease of needle puncture and particularly low complication rates (pseudo-aneurysm, infection, and thrombosis). However, AV grafts still require time for the graft material to mature prior to use, so that a temporary access device, such as a Quinton catheter, must be inserted into a patient for hemodialysis access until the AV graft has matured. The use of temporary catheter access exposes the patient to additional risk of bleeding and infection, as well as discomfort. Also, patency rates of ePTFE access grafts are still not satisfactory, as the overall graft failure rate remains high. Sixty percent of these grafts fail yearly, usually due to stenosis at the venous end. (See Besarab, A & Samararpungavan D., “Measuring the Adequacy of Hemodialysis Access”. Curr Opin Nephrol Hypertens 5(6) 527-531, 1996, Raju, S. “PTFE Grafts for Hemodialysis Access”. Ann Surg 206(5), 666-673, Nov. 1987, Koo Seen Lin, L C & Burnapp, L. “Contemporary Vascular Access Surgery for Chronic Hemodialysis”. J R Coll Surg 41, 164-169, 1996, and Kumpe, D A & Cohen, M A H “Angioplasty/Thrombolytic Treatment of Failing and Failed Hemodialysis Access Sites: Comparison with Surgical Treatment”. Prog Cardiovasc Dis 34(4), 263-278, 1992, all herein incorporated by reference in their entirety). These failure rates are further increased in higher-risk patients, such as diabetics. These access failures result in disruption in the routine dialysis schedule and create hospital costs of over $2 billion per year. (See Sharafuddin, M J A, Kadir, S., et al. “Percutaneous Balloon-assisted aspiration thrombectomy of clotted Hemodialysis access Grafts”. J Vasc Interv Radiol 7(2) 177-183, 1996, herein incorporated by reference in its entirety).

### SUMMARY OF THE INVENTION

Vascular access systems for performing hemodialysis are disclosed. One embodiment relates to vascular access systems comprising graft material reinforced with expanded PTFE to resist stretching of the graft material and thereby resist leakage associated with stretched or bent graft material. Another embodiment of the invention relates to vascular access systems having auxiliary access lumens that may be sealed and removed from the primary portion of the vascular access system. Other embodiments relate to vascular access grafts comprising an instant access material reinforced with expanded PTFE to resist stretching of the instant access material and thereby resist leakage associated with stretching or bending. The graft may comprise two end segments comprising ePTFE without the instant access material to allow easier anastomosis of the graft to veins and arteries. The graft may have a unibody design or have modular components that may be joined together to create a graft with customized length or other features. One or more sections of the graft may also be cut or trimmed to a custom length.

In one embodiment, a biocompatible graft material is provided, comprising a leak-resistant layer bonded to a stretch-resistant structure, wherein the stretch-resistant structure prevents expansion of the leak-resistant layer that would substantially result in opening and leakage of any needle puncture sites in the leak-resistant layer. The leak-resistant layer may comprise silicone. The silicone may be silicone tubing. The silicone tubing may be everted silicone tubing. The stretch-resistant structure may be a stretch-resistant layer bonded to the leak-resistant layer. The stretch-resistant layer may comprise ePTFE. The ePTFE may have an internodal spacing of about 25 microns to about 30 microns.

In another embodiment, an implantable fluid conduit is provided, comprising a first conduit having a first end, a second end, a lumen therebetween, and a connector with an opening contiguous with the lumen of the first conduit, wherein the first end and second end adapted to interface with a body fluid conduit; and a second conduit having an elastic first end, a second end and a lumen therebetween, wherein the elastic first end of the second conduit may be disengageably connected to the connector of the first conduit. The implantable fluid conduit may further comprise a conduit pressurizer, the conduit pressurizer comprising a distal tip configured to engage the second end of the second conduit, a plug configured to seal the lumen of the second conduit, and a volume of fluid configured to propel the plug from the distal tip of the conduit pressurizer to about the first end of the second conduit. The implantable conduit pressurizer may be a syringe. The conduit pressurizer may be a fluid pump.

In another embodiment, an implantable fluid conduit is provided, comprising a first conduit having a first end, a second end, a lumen therebetween, and a connector with an opening contiguous with the lumen of the first conduit, wherein the first end and second end adapted to interface with a body fluid conduit; a second conduit having an first end, a second end and a lumen therebetween, wherein the first end of the second conduit may be connected to the connector of the first conduit, and wherein the first end of the second conduit has a pressure responsive reduced configuration and an expanded configuration, wherein the first end of the second conduit may be configured to change from the pressure responsive reduced configuration the expanded configuration with increased pressure within the lumen of the second conduit. The implantable fluid conduit may further comprise a conduit pressurizer, the conduit pressurizer comprising a distal tip configured to engage the second end of the second conduit, a plug configured to seal the lumen of the second conduit, and a volume of fluid configured to propel the plug from the distal tip of the conduit pressurizer to about the first end of the second conduit.

In another embodiment, a syringe for sealing catheters is provided, comprising a distal tip configured to sealably connect to an end of a catheter, a plug configured to seal a lumen of said catheter, and a volume of pressurizable fluid proximal to the plug configured to propel the plug into said catheter.

In another embodiment, a kit for treating a patient is provided, comprising a vascular access system, a syringe having a tip, and a pre-formed plug configured to reside in the tip of the syringe.

In another embodiment, a method for treating a patient is provided, comprising providing a first conduit having a first end, a second end, a lumen therebetween, and a connector with an opening contiguous with the lumen of the first conduit, wherein the first end and second end adapted to interface with a body fluid conduit; and a second conduit having an elastic first end, a second end and a lumen therebetween, wherein the elastic first end of the second conduit is disengageably connected to the connector of the first conduit; and attaching the first end of the first conduit to a body conduit of a patient and the second end of the first conduit to a second body conduit of the patient while positioning the second end of the second conduit outside the patient. The method may further comprise detaching the second conduit from the first conduit and removing the second conduit from the patient. The method may further comprise sealing off the second conduit from the first conduit by propelling a plug into the first conduit using a syringe.

In one embodiment, a biocompatible graft is provided, comprising a leak-resistant layer bonded to a stretch-resistant structure, wherein the stretch-resistant structure prevents expansion of the leak-resistant layer that would substantially result in opening and leakage of any needle puncture sites in the leak-resistant layer. The leak-resistant layer may comprise a silicone layer and the stretch-resistant layer may comprise an ePTFE layer. The leak-resistant layer may comprise a leak-resistant tubing material and the stretch-resistant layer may comprise stretch-resistant tubing material. The ePTFE layer may be ePTFE tubing comprising a length, an exterior surface, an outer diameter, a first end, a second end, a lumen therebetween, and a inner diameter. The silicone layer may comprise silicone tubing having a first end and a second end. The silicone tubing may be applied to the exterior surface of the ePTFE tubing or to the lumen of the ePTFE tubing. The silicone tubing may be everted silicone tubing. The silicone tubing may have a length less than the length of the ePTFE tubing. The biocompatible graft may further comprise a layer of ePTFE overlayed on the silicone tubing. The overlayed layer of ePTFE may completely cover the silicone tubing. The silicone tubing may be located at least about 0.25 cm, 0.5 cm or 1 cm from the first end of the ePTFE tubing. The silicone tubing may be located at least about 0.25 cm, 0.5 or 1 cm from the second end of the ePTFE tubing. The lumen of the ePTFE tubing may comprise a luminal smaller diameter zone, a luminal transition zone and a luminal larger diameter zone. The silicone tubing may be applied to the lumen of the ePTFE tubing about the luminal transition zone and the luminal larger diameter zone. The exterior surface of the ePTFE tubing may comprise an exterior smaller diameter zone, an exterior transition zone and an exterior larger diameter zone. The silicone tubing may be applied to the exterior surface of the ePTFE tubing. The silicone tubing may be applied at least to the exterior surface of the ePTFE tubing about the luminal transition zone and the luminal smaller diameter zone. The leak-resistant layer and stretch-resistant layer may form an instant access segment located between a first ePTFE end segment and a second ePTFE end segment. The first ePTFE end segment and instant access segment may be integrally formed or may bejoined by a segment connector. The biocompatible graft may further comprise an anti-kink structure about the first end of the silicone tubing or the second end of the silicone tubing, or anti-kink structures about both the first end of the silicone tubing and the second end of the silicone tubing. The biocompatible graft may also comprise a separation member embedded generally within the silicone tubing or between the silicone tubing and the ePTFE tubing. The separation member may be a helical unwinding member.

In another embodiment, a method for treating a patient is provided, comprising providing an implantable medical device comprising a silicone layer bonded to an ePTFE layer, wherein the ePTFE layer may be configured to prevent stretching of the silicone layer to a degree that opens any puncture hole in the silicone layer sufficient to allow passage of fluid in a body conduit; and attaching the implantable medical device to a body conduit. The implantable medical device may comprise a vascular access graft or vascular access port.

In another embodiment, a method for implanting a vascular graft is provided, comprising providing a biocompatible graft having a first end segment, an instant access segment and a second end segment; attaching one of the end segments to an artery; and attaching the other end segment to a vein; wherein the instant access segment may comprise a leak-resistant structure bonded to a stretch-resistant structure. The leak-resistant structure may be a tubular structure of leak-resistant material. The stretch-resistant structure may be a tubular structure of stretch-resistant material. The leak-resistant structure may comprise a leak-resistant material having a longitudinal length of at least about 5 cm, 7 cm, 9 cm or 11 cm. The longitudinal length may be contiguous. The leak-resistant structure may comprise a silicone layer bonded to the stretch-resistant structure, the stretch-resistant structure comprising ePTFE or PTFE. The method may further comprise attaching one of the end segments and the instant access segment using a connector, or attaching one of the end segments and the instant access segment using a means for connecting vascular access segments. One of the end segments and the instant access segment may be integrally formed during manufacture. The method may further comprise cutting the instant access segment into a first instant access subsegment and a second instant access subsegment. The method may further comprise attaching one of the instant access subsegments to one of the end segments. The remaining subsegment may be discarded. The instant access segment may further comprise a separation member located generally within the leak-resistant structure or between the leak-resistant structure and the stretch-resistant structure. The method may further comprise cutting the instant access segment and/or applying force to the separation member to at least partially separate a portion of the leak-resistant structure from the stretch-resistant structure. The method may further comprise removing the at least partially separated portion of the leak-resistant structure to form the second end segment from a portion of the instant access segment. The first end segment may have a smaller diameter than the second end segment, or the first end segment and the second end segment may have smaller diameters than the instant access segment.

In another embodiment, an implantable vascular access graft designed for rapid access to blood flow through the graft when the graft is implanted in a patient is provided, said graft comprising a polyurethane tube, having an inside surface, an outside surface and a length extending from a first end to a second end; and a structure resistant to leakage after puncture by a needle, said structure comprising a layer attached to said tube around said inside or outside surface and extending less than the length of said tube between said first and second ends, so as to provide section of said tube free of said structure at the ends of said tube.

In one embodiment, a biocompatible graft is provided, comprising a leak-resistant layer bonded to a stretch-resistant structure, wherein the stretch-resistant structure resists expansion of the leak-resistant layer that would substantially result in opening and leakage of any needle puncture sites in the leak-resistant layer, and wherein the leak-resistant layer has an everted configuration. The leak-resistant layer may comprise a silicone layer and the stretch-resistant layer may comprise an ePTFE layer, or the leak-resistant layer may comprise a leak-resistant tubing material and the stretch-resistant layer may comprise stretch-resistant tubing material. The ePTFE layer may be ePTFE tubing comprising a length, an exterior surface, an outer diameter, a first end, a second end, a lumen therebetween, and a inner diameter. The silicone layer may comprise silicone tubing may have a first end and a second end. The silicone tubing may be applied to the exterior surface of the ePTFE tubing and/or the lumen of the ePTFE tubing. The silicone tubing may have a length less than the length of the ePTFE tubing. The biocompatible graft may further comprise a layer of ePTFE overlayed on the silicone tubing. The overlayed layer of ePTFE may completely cover the silicone tubing. The silicone tubing may be located at least about 0.25 cm from the first end of the ePTFE tubing, or at least about 0.5 cm from the first end of the ePTFE tubing, or at least about 0.25 cm from the second end of the ePTFE tubing, or at least about 1 cm from the first end of the ePTFE tubing. The silicone tubing may be located at least about 0.5 cm from the second end of the ePTFE tubing, or at least about 1 cm from the second end of the ePTFE tubing. The lumen of the ePTFE tubing may comprise a luminal smaller diameter zone, a luminal transition zone and a luminal larger diameter zone. The silicone tubing may be applied to the lumen of the ePTFE tubing about the luminal transition zone and the luminal larger diameter zone. The exterior surface of the ePTFE tubing may comprise an exterior smaller diameter zone, an exterior transition zone and an exterior larger diameter zone. The silicone tubing may be applied at least to the exterior surface of the ePTFE tubing about the luminal transition zone and the luminal smaller diameter zone. The silicone tubing may be applied to the exterior surface of the ePTFE tubing. The leak-resistant layer and stretch-resistant layer may form an instant access segment located between a first ePTFE end segment and a second ePTFE end segment. The first ePTFE end segment and instant access segment may be integrally formed. The first ePTFE end segment and instant access segment may be joined by a segment connector. The biocompatible graft may further comprise at least one anti-kink structure about the first end of the silicone tubing or the second end of the silicone tubing. The biocompatible graft may further comprise anti-kink structures about both the first end of the silicone tubing and the second end of the silicone tubing. The biocompatible graft may further comprise a separation member embedded generally within the silicone tubing, or between the silicone tubing and the ePTFE tubing. The separation member may be a helical unwinding member. The leak-resistant layer may be longitudinally compressed.

In one embodiment, a hemodialysis graft is provided, comprising an everted elastomeric tubular structure. The hemodialysis graft may further comprise a tubular graft material bonded to the everted elastomeric tubular structure.

In one embodiment, biocompatible vascular graft is provided, comprising .a tubular leak-resistant material having an outer surface, an inner surface, a first end, a second end, a longitudinal axis, and an inner lumen between the first end and the second end, wherein at least a portion of the tubular leak-resistant material is circumferentially compressed. The tubular leak-resistant material may be axially compressed and/or radially compressed. The radial compression of the tubular leak-resistant material may be inherent in the tubular leak-resistant material. The outer surface of the tubular leak-resistant material about may have a circumferential tension that radially compresses the tubular leak-resistant material about the inner surface of the tubular leak-resistant material. The tubular leak-resistant material may exhibit increasing compression from its outer surface to its inner surface. The outer surface of the tubular leak-resistant material may be in an expanded configuration and the inner surface of the tubular leak-resistant material may be in a compressed configuration. The tubular leak-resistant material may be an everted tubular material. The tubular leak-resistant material may be a silicone tube or a polyurethane tube. The biocompatible graft may further comprise a radial compression structure. The radial compression structure may be a tubular compression structure. The biocompatible graft may further comprise one or more stretch-resistant structures joined to the tubular leak-resistant material and configured to resist stretching of the tubular leak-resistant material. The one or more stretch-resistant structures may comprise a plurality of stretch resistant structures embedded within the tubular leak-resistant material. The plurality of stretch resistant structures may be discrete fibers or strands. The one or more stretch-resistant structures may comprise a stretch resistant tube concentrically arranged with the tubular leak-resistant material. The stretch resistant tube may be bonded to outer surface of the tubular leak resistant material. The stretch resistant tube may be bonded to inner surface of the tubular leak resistant material. The stretch resistant tube may be an ePTFE tube. The stretch-resistant material may be ePTFE. The eTPFE has an average internodal distance of about 25 microns to about 30 microns along the longitudinal axis of the tubular leak-resistant material. The compression of the tubular leak-resistant material may be radial.

In one embodiment, a method for manufacturing a vascular graft is provided, comprising everting a resilient polymeric tube; and bonding together a stretch resistant structure and the resilient polymeric tube. The resilient polymeric tube may be a silicone tube. The stretch resistant structure may be a stretch resistant graft structure. The stretch resistant graft structure may comprise ePTFE. The stretch resistant structure has a tubular configuration. The stretch resistant structure may be bonded to an outer surface of the resilient polymeric tube. The method for manufacturing a vascular graft may further comprise disposing the everted resilient polymeric tube over a tubular graft. The tubular graft, everted resilient polymeric tube and stretch resistant structure may each have a length and wherein the length of the stretch resistant structure may be shorter than the length of the tubular graft. The stretch resistant structure may be longitudinally compressible. The method for manufacturing a vascular graft may further comprise disposing a tubular graft onto an outer surface of the resilient polymeric tube prior to everting the resilient polymeric tube, wherein everting the resilient polymeric tube also everts the tubular graft.

In one embodiment, a method for treating a patient is provided, comprising providing an implantable medical device comprising an everted silicone layer bonded to an ePTFE layer, wherein the ePTFE layer is configured to prevent stretching of the silicone layer to a degree that opens any puncture hole in the silicone layer sufficient to allow passage of fluid in a body conduit; and attaching the implantable medical device to a body conduit. The implantable medical device may comprise a vascular access graft. The implantable medical device may comprise a vascular access port.

In one embodiment, a method for implanting a vascular graft is provided, comprising providing a biocompatible graft having a first end segment, an instant access segment and a second end segment; attaching one of the end segments to an artery; and attaching the other end segment to a vein; wherein the instant access segment comprises an everted leak-resistant structure bonded to a stretch-resistant structure. The everted leak-resistant structure may be a tubular structure of leak-resistant material. The everted leak-resistant structure may be longitudinally compressible. The stretch-resistant structure may be a tubular structure of stretch-resistant material. The leak-resistant structure may be may comprise a leak-resistant material having a continuous or a net longitudinal length of at least about 5 cm, at least about 7 cm, at least about 9 cm, or at least about 11 cm. The leak-resistant structure may comprise a silicone layer bonded to the stretch-resistant structure, the stretch-resistant structure comprising ePTFE or PTFE. The method may further comprise attaching one of the end segments and the instant access segment using a connector. The method may further comprise attaching one of the end segments and the instant access segment using a means for connecting vascular access segments. One of the end segments and the instant access segment may be integrally formed during manufacture. The method may further comprise cutting the instant access segment into a first instant access subsegment and a second instant access subsegment. The method may further comprise attaching one of the instant access subsegments to one of the end segments. The instant access segment further may comprise a separation member generally located within the leak-resistance structure or between the leak-resistant structure and the stretch-resistant structure. The method may further comprise cutting the instant access segment. The method may further comprise applying force to the separation member to at least partially separate a portion of the leak-resistant structure from the stretch-resistant structure. The method for implanting a vascular graft as in claim 86, may further comprise removing the at least partially separated portion of the leak-resistant structure to form the second end segment from a portion of the instant access segment. The first end segment may have a smaller diameter than the second end segment, or the first end segment and the second end segment may have smaller diameters than the instant access segment.

In one embodiment, an implantable vascular access graft designed for rapid access to blood flow through the graft when the graft may be implanted in a patient is provided, said graft comprising a polyurethane tube, having an inside surface, an outside surface and a length extending from a first end to a second end; and a structure resistant to leakage after puncture by a needle, said structure comprising a layer attached to said polyurethane tube around said inside or outside surface and extending less than the length of said tube between said first and second ends, so as to provide section of said tube free of said structure at the ends of said tube.

### BRIEF DESCRIPTION OF THE DRAWINGS

The structure and method of using the invention will be better understood with the following detailed description of embodiments of the invention, along with the accompanying illustrations, in which:

FIG. 1A is a cross-sectional schematic view of one embodiment of the connector. FIGS. 1B and 1C depict the connector edges of the connector in FIG. 1A.

FIG. 2A is an exploded view of one embodiment of the connector system; FIG. 2B is a cross-sectional view of the connector system in FIG. 2A when assembled.

FIG. 3 is an elevational view of one embodiment of the invention comprising a multi-component vascular access system with an access region of self-sealing material.

FIG. 4 is a schematic representation of a vascular access system with a transcutaneous port.

FIG. 5 is an elevational view of a graft section with an anti-kink support.

FIGS. 6A and 6B are schematic elevation and cross-sectional views, respectively, of one embodiment of a catheter section with embedded reinforcement.

FIGS. 7A to 7C are detailed elevational views of one embodiment of a catheter section reinforced with a removably bonded filament. FIG. 7B depicts the removal of a portion of the filament from FIG. 7A. FIG. 7C illustrates the catheter section of FIGS. 7A and 7B prepared for fitting to a connector.

FIGS. 8A to 8F are schematic representations of one embodiment of the invention for planting a two-section vascular access system.

FIGS. 9A to 9E are schematic representations of another embodiment of the invention for implanting a two-section vascular access system.

FIG. 10 is a schematic representation of a self-sealing conduit comprising multiple layers.

FIG. 11 is a schematic representation of a vascular access system with an attached temporary catheter.

FIGS. 12A and 12B are detailed schematic representations of vascular access system coupled to a temporary catheter using a compressive interface.

FIG. 13 is a cross-sectional view of a connector with biased flaps for providing access to the blood passageway.

FIGS. 14A and 14B are schematic cross-sectional views of a conduit connector with a pair of mechanical valves for attaching a temporary catheter in the open and closed configurations, respectively.

FIGS. 15A to 15C are schematic representations of a temporary catheter with a full-length plug.

FIGS. 16A to 16C are schematic representations of a locking temporary catheter used with a proximal plug and catheter cutter.

FIGS. 17A to 17D are schematic representations of a vascular access system with an auxiliary catheter and hydraulic removal system.

FIG. 18 is a schematic cross-sectional view of an immediate-access graft device.

FIG. 19 is a schematic cross-sectional view of another immediate-access graft device.

FIG. 20 is a schematic cross-sectional view of another immediate-access graft device.

FIG. 21 is a schematic cross-sectional view of another immediate-access graft device.

FIG. 22 is a schematic cross-sectional view of another immediate-access graft device.

FIG. 23 is a schematic elevational view of another immediate-access graft device.

FIG. 24 is a schematic elevational view of a multi-section immediate-access graft device with a connector.

FIGS. 25A and 25b are schematic cross-sectional views of a silicone tube structure before and after eversion.

FIGS. 26A and 26b are schematic cross-sectional views of a silicone tube structure compressed into the inner lumen of a compression tube.

FIG. 27A is a table depicting the predicted strain in an everted silicone tube. FIG. 7B is a chart illustrating the predicted percentage of material strain in the everted tube.

FIG. 28 is a graph depicting the stretch-resistant property of ePTFE.

### DETAILED DESCRIPTION OF THE PREFERRED EMBODIMENT

Research indicates that graft failures from localized stenosis at the venous end of AV grafts are primarily due to intimal hyperplasia, compliance mismatch between the graft and the native vein anastomosis, and turbulent flow at the anastomosis site. Kanterman R. Y. et al “Dialysis access grafts: Anatomic location of venous stenosis and results of angioplasty.” Radiology 195: 135-139, 1995. We hypothesize that these causes could be circumvented by eliminating the venous anastomosis and instead, using a catheter to discharge the blood directly into the venous system. We have developed vascular access system that eliminates the venous anastomosis in the AV shunt, using a catheter element at the venous end and a synthetic graft element anastomosed to the artery in the standard fashion. We believe that such system should eliminate or reduce venous hyperplasia, which is the largest reason for AV shunt failure.

A. Vascular Access System (VAS)

Although these devices may be may be constructed as a single-piece, integrated device, a multi-piece device comprising separate components that are later joined together may also be designed. A multi-component device may have several advantages. First, a multi-piece device allows switch-out of one or more components of the device. This allows the tailoring of various device characteristics to the particular anatomy and/or disease state, for instance, by using components of different dimensions. This also reduces the cost of treating patients in several ways. It reduces the amount of inventory of a given device by stocking an inventory range of components, rather than an inventory range of complete devices. Also, if an incorrect device is initially selected for use in a patient, only the incorrect component is discarded, rather than the entire device. Second, separate multiple components of a device may be easier to manufacture compared to an integrated form of the device. Third, it may be easier for a physician to implant separate components of a device and then join them together rather than implanting an integrated device. Fourth, it allows the components to be trimmable as needed to accommodate various patient anatomies. An integrated device may be excessively bulky and can slow the implantation procedure, thereby increasing operating room time and costs as well as increasing the risk of physician error.

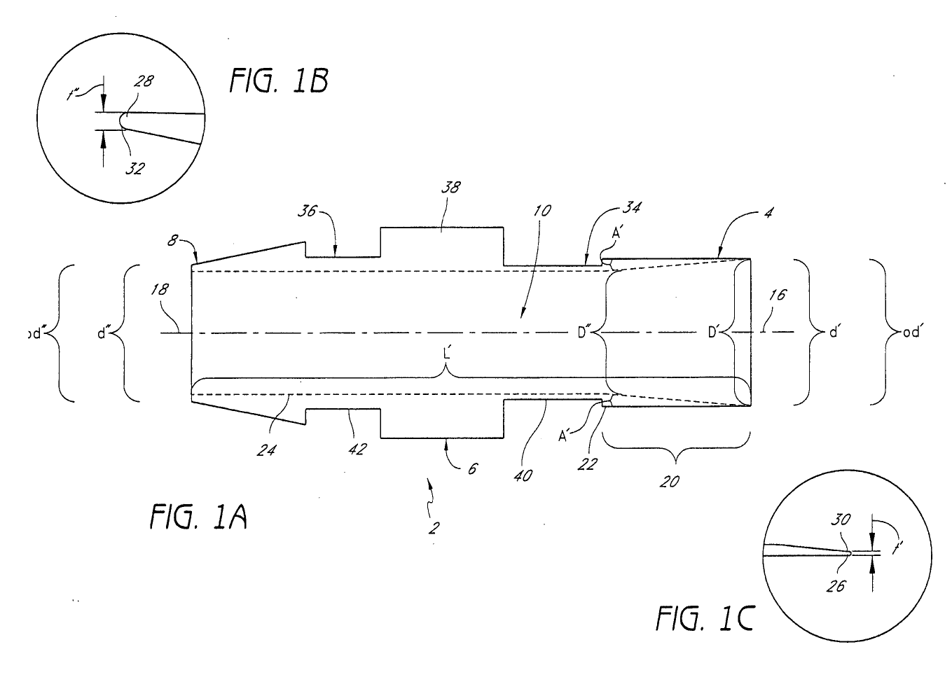

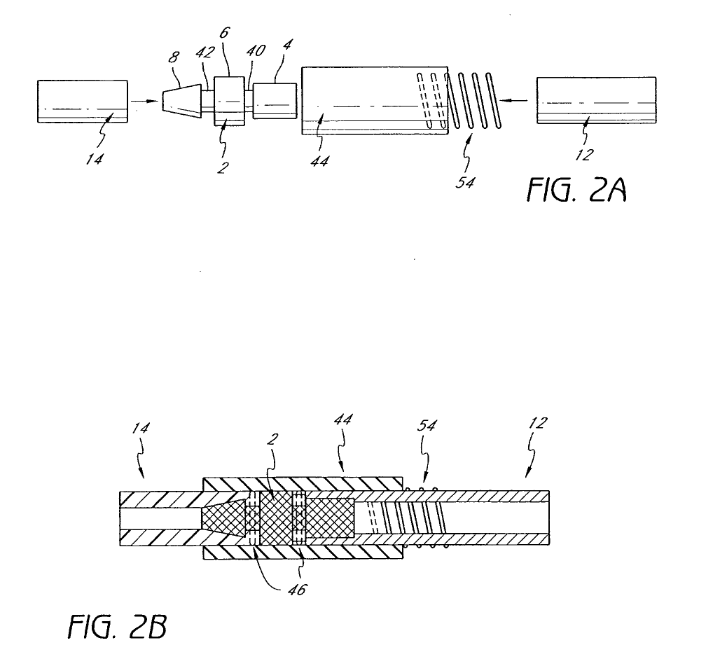

FIG. 1 depicts one embodiment of the invention. The invention comprises a connector 2 having a first end 4 for connecting to a first fluid conduit, a middle portion 6 and a second end 8 for connecting to a second fluid conduit, and a lumen 10 from the first end to the second end. Referring to FIGS. 2A and 2B, the first fluid conduit 12 is typically a hemodialysis graft component while the second fluid conduit 14 is typically a catheter, but other combinations may also be used, such as graft/graft, catheter/graft or catheter/catheter.

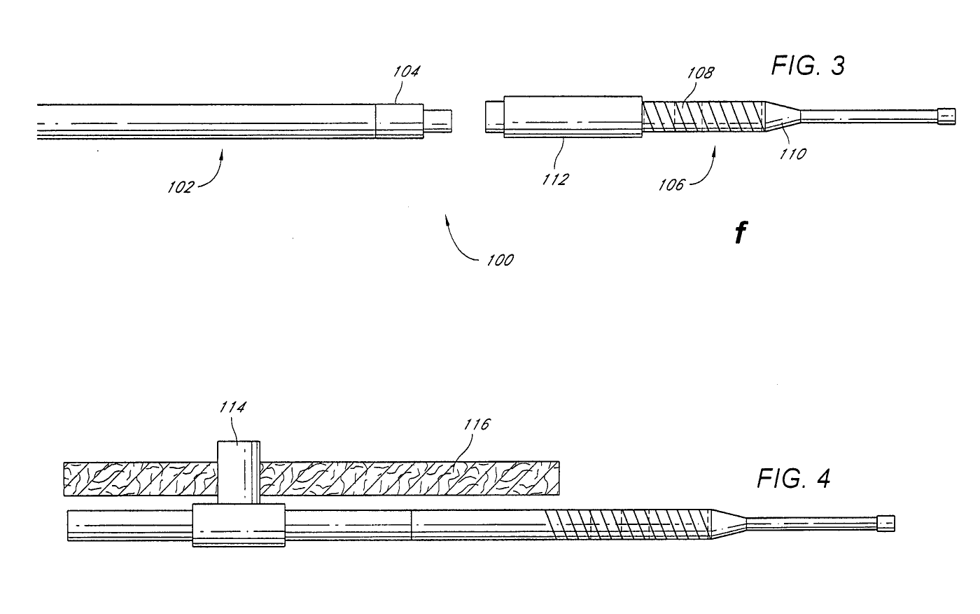

In the one embodiment of the invention, depicted in FIG. 3, the vascular access system (VAS) 100 comprises a first section 102 of graft material with an integrated connector end 104 attachable to a second section 106 comprising a catheter component that is adapted to transport the blood and also to be inserted into the venous system using a venotomy or even less-invasive procedure. The second section 106 may have a small diameter of about 7 mm or less, preferably about 6 mm or less, and most preferably about 5 mm or less so it does not require a large venotomy to implant the second section 106 and whereby the second section 106 does not occupy an excessive amount of space in the venous system. The VAS 100 preferably has thin walls to maximize the area available to flow through the VAS 100, which may be achieved using reinforced thin-wall tubing. The second section 106 has an opening adapted to be within the vein itself and wherein the opening is distant or is located downstream from the insertion site where the second section 106 inserts into the vein. The portion of the second section 106 insertable into the vein has an outer diameter which is less than an inner diameter of the vein in which it is disposed such that, in operation, blood can flow through the second section into the vein and also through the vein itself around the outer surface of the second section 106. The second section 106 may be adapted to be entirely subcutaneous in use and configured to avoid, in use, a blood reservoir therein and to provide continuous blood flow. The selection of the diameter and length of the two sections 102, 106 may be determined by assessing the vein in which the VAS 100 is to be inserted, the insertion length of the second section 106, and/or possibly the flow rate and pressure drop criteria needed to perform hemodialysis.

The second section 106 may be trimmed and then attached to the graft section 102 to achieve the desired total length. The graft and catheter sections 102, 106 are made to resist kinking and crushing, yet not be excessively stiff. In one embodiment of the invention, these properties may be provided by a spiral reinforcement 108 in a silicone tubing 110. Other materials that may be used include PTFE, polyurethane and other hemocompatible polymers. Also shown in FIG. 3 is a section of the catheter element 106 comprising a self-sealing area 112 that provides access by needles to perform dialysis either temporarily while the graft 102 is healing in or on a long-term basis. The self-sealing area 112 is preferably self-supported (e.g. frameless), generally having the same diameter and shape as the catheter and/or graft sections of the VAS, generally having a tubular configuration so that is may be punctured at any point along its length and/or circumference. The self-sealing area 112 may comprise a self-sealing material that forms a layer of the wall of at least a portion of the graft and/or catheter section of the VAS. Unlike self-sealing material provided in an access port, the self-sealing area 112 remains flexible along its length or longitudinal axis to facilitate implantation of the VAS and also to provide a longer self-sealing area 112 than can be provided by a self-sealing region on a bulky access port. The longer length allows the insertion of dialysis needles within a larger surface area so that the same small skin region need not be repeatedly pierced and thereby significantly reducing the chance of forming a sinus tract, which could lead to infection and/or bleeding. This also allows a given needle tract more time to recover between needle piercings, and therefore may further reduce the risk of infection and/or bleeding compared to traditional access ports. In one embodiment, the self-sealing area 112 has a length of at least about 2 inches, in other embodiments at least about 3 inches, and in still other embodiments, at least about 4 inches or 5 inches. The VAS may also optionally comprise a flow sensor that is imbedded in the wall of the VAS which can be interrogated externally to give a reading of flow in the device, and/or a section of tubing that can be adjusted post implant to control flow. These and other features are described in greater detail below.

Other access sites may be provided using one or more other components, structures or materials, including the use a puncture-resistant, circumferentially compressed tubing material in a portion of or all of the catheter section, a gel material sandwiched within the walls of the tubing, a low durometer material, a needle-accessible graft section or any combination thereof, an implantable port than can be accessed by needles, and/or a transcutaneous port 114 accessible without piercing the skin 116, as depicted in FIG. 4. Some of these features are discussed in greater detail below.

In some embodiments of the invention, the graft and/or catheter sections may also be coated with one or more therapeutic agents to address any of a variety of VAS-related effects, including but not limited to resisting thrombosis, reducing infection, speeding up healing time, promoting cell growth and/or improving arterial anastomosis. These agents include but are not limited to heparin, carbon, silver compounds, collagen, antibiotics, and anti-restenotic agents such as rapamycin or paclitaxel. These agents may be bonded to a surface of the VAS, as is known in the art, with heparin and chlorhexidine-bonded materials, or these agents may be eluted from a drug-eluting polymer coating.

Similarly, the porosity and other characteristics of the self-sealing area 112 may also be altered to augment its effects. For example, this can be done by varying the porosity, construction and wall thickness of the conduit material. Some commonly used materials are ePTFE, polyurethane, silicone or combinations of these materials manufactured in such a way as to render the outer wall surface of the conduit porous. The porous nature facilitates tissue in-growth, which can help to reduce infection rates. It is believed that a porosity of about 20 μm or less in a material provides leak-resistance of the bulk material before needle puncture. Therefore it is preferred but not required that at least a portion of the wall thickness be constructed of a material with a porosity of about 20 μm or less. However, porosities of about 10 μm to about 1000 μm or more on the outer surface may facilitate cellular ingrowth into a porous surface that will reduce serous fluid accumulation surrounding the implant, which in turn reduces the infection rate associated with needle puncture. More preferably, porosities of about 20 μm to about 200 μm, and most preferably about 100 μm to about 200 μm are used. To provide a material that is leak-resistant and has improved cellular ingrowth, a multi-layer material may be provided, with a surface layer having a porosity and/or or other features for facilitating cellular ingrowth, and a subsurface material with features for facilitating leak-resistance. However, that cellular-ingrowth may also be achieved with smooth-surface devices through the use of various substrates or therapeutic agents coated onto the graft and/or catheter section. Furthermore, in regions of the VAS not intended for needle puncture, those regions may be provided with a porous layer or coating to facilitate tissue ingrowth without requiring a leak-resistant sub-layer. These materials are also biocompatible and may be manufactured, for example, so that they have a comparable compliance to the arteries to which they are attached to facilitate the creation and patency of the arterial anastomosis. The inner and outer surfaces of the conduit may also be of different materials, surface structure, and possess coatings to enhance reactions with the body such as patency, infection resistance, and tissue ingrowth.

1. Graft Section

The graft section of the vascular access system may comprise ePTFE, polyurethane, silicone, Dacron® or other similar material. The graft section 102 of the VAS 100 may have a length of at least about 20 cm, preferably greater than about 40 cm, and most preferably greater than about 60 cm. The graft section 102 may have an inside diameter within the range of from about 5.5 mm to about 6.5 mm, and sometimes about 5 mm to about 7 mm. The wall thickness of the graft section 102 may be about 0.3 mm to about 2 mm, sometimes about 0.4 mm to about 1 mm, and preferably about 0.5 mm to about 0.8 mm.

As mentioned previously, strain relief is provided in some embodiments of the invention. Strain relief may be advantageous for conduits or grafts that comprise PTFE or other flexible materials and may prevent occlusion of the conduit or graft. The strain relief structure typically comprises a flexible spiral or coil that extends from an end of the connector or connector sleeve and onto the outer surface of or within the wall of the conduit/graft. The strain relief structure may comprise a biocompatible metal or plastic.

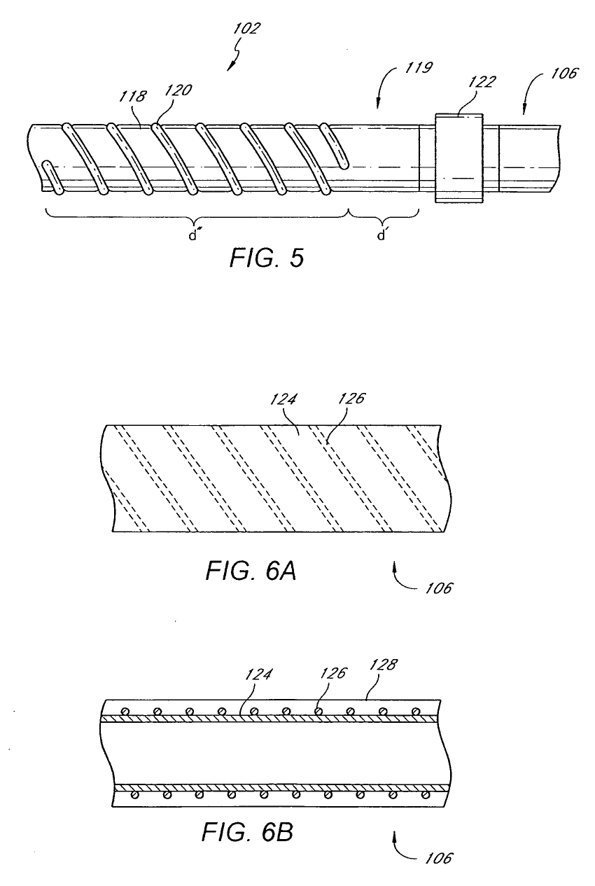

In an alternate embodiment of the invention, rather than providing a strain relief structure projecting from the connector or connector sleeve onto the graft section, the strain relief structure may be attached directly to the graft section. In one particular embodiment depicted in FIG. 5, the graft section 102 comprises ePTFE material 118 with a PTFE spiral strain relief structure 120 generally located at the connector end 119 of the graft section 102 that is attached or attachable to the catheter section 106 or conduit connector 122 of the vascular access system (VAS) 100. The embodiment depicted in FIG. 5 is a spiral strain relief structure 120, but one of ordinary skill in the art will understand that other strain relief structures may also be attached to the graft section 102. In some instances, the spiral PTFE support is configured to terminate generally at the connector end of the graft section, while in other embodiments, the spiral strain relief structure may extend beyond the end of the graft section to contact the connector or connector sleeve. In other embodiments, the spiral PTFE support is spaced within about 0.2 cm from the connector end 119 of the graft section 102. The spiral PTFE support may have a length of about 1 cm to about 8 cm, preferably about 2 cm to about 6 cm, and most preferably about 2 cm to about 4 cm. The spiral PTFE support may be staked (cold, heat, thermal, and/or ultrasonic) to the PTFE graft material, bonded to the graft material using an adhesive, or held in place by a coating on the graft section 102.

In another embodiment, the graft material is coated and/or embedded with silicone or other elastic material in the region near the connector to improve contact of the wall of the graft with the connector when graft is subjected to bending. This may be beneficial because the ePTFE graft material is naturally plastically deformable and, when it is subjected to a bend at the end of the connector, it may open up a gap that will disrupt blood flow (causing turbulence and pooling) and result in clot formation. The addition of elastic material may help maintain a tighter fit between the graft and connector surface. In one preferred embodiment, the graft is spray or dip coated using a silicone-xylene blend having a viscosity of approximately 200 cps. The viscosity may range from about 50 to about 1000 cps, more preferably about 100 to about 300 cps, and most preferably from about 150 to about 250 cps. Alternatives include low viscosity silicones, urethanes, styrenic block copolymers or other elastomers without solvents or with xylenes, toluenes, napthas, ketones, THF or other suitable miscible solvents.

The graft section of the VAS may optionally have length markers on its surface to facilitate trimming of the graft section to a desired length for individualizing the device to a particular patient's anatomy. The length markers or other markers provided in the graft section may also be radio-opaque to facilitate radiographic visualization of the graft section.

2. Catheter Section

As previously mentioned, the catheter section of the VAS may comprise a conduit having a non-uniform diameter. The end of the catheter section adapted for insertion into a vein or other blood vessel may have an inside diameter of about 3 mm to about 10 mm, sometimes within the range of about 4 mm to about 6 mm, and preferably about 5 mm, and may have an embedded or external spiral support to provide kink resistance. The end of the catheter section adapted for attachment to a connector or graft section may have a larger diameter because it does not reside within the lumen of a blood vessel. The selection of the inner diameter, outer diameter and length of the catheter section may be selected by one skilled in the art, based upon factors including but not limited to the vein into which the second body fluid segment is being inserted into, the length of catheter to be inserted through the vein wall, as well as the desired flow rate and fluid resistance characteristics.

The catheter section typically comprises PTFE, polyurethane or silicone. Other biocompatible materials that may be used include polyethylene, homopolymers and copolymers of vinyl acetate such as ethylene vinyl acetate copolymer, polyvinylchlorides, homopolymers and copolymers of acrylates such as polymethylmethacrylate, polyethylmethacrylate, polymethacrylate, ethylene glycol dimethacrylate, ethylene dimethacrylate and hydroxymethyl methacrylate, polyurethanes, polyvinylpyrrolidone, 2-pyrrolidone, polyacrylonitrile butadiene, polycarbonates, polyamides, fluoropolymers such as homopolymers and copolymers of polytetrafluoroethylene and polyvinyl fluoride, polystyrenes, homopolymers and copolymers of styrene acrylonitrile, cellulose acetate, homopolymers and copolymers of acrylonitrile butadiene styrene, polymethylpentene, polysulfones, polyesters, polyimides, polyisobutylene, polymethylstyrene, biocompatible elastomers such as medical grade silicone rubbers, polyvinyl chloride elastomers, polyolefin homopolymeric and copolymeric elastomers, styrene-butadiene copolymers, urethane-based elastomers, and natural rubber or other synthetic rubbers, and other similar compounds known to those of ordinary skilled in the art. See Polymer Handbook, Fourth Edition, Ed. By J. Brandup, E. H. Immergut, E. A. Grulke and D. Bloch, Wiley-Interscience, NY, Feb. 22, 1999.

Preferably the portion of the catheter section that is insertable into the vein is sized to allow collateral flow of blood around the inserted catheter and through the vascular site where the catheter section is inserted. It is also preferred in some embodiments that the catheter section of the VAS be dimensioned to allow percutaneous insertion of the catheter section into a vein using the Seldinger technique, rather than by venous cutdown or full surgical exposure of the vein. Percutaneous insertion of the catheter section into a vein, such as an internal jugular vein, for example, is facilitated by a catheter section having an outer diameter of no greater than about 6 mm, and preferably no greater than about 5 mm or about 4 mm.

In one embodiment of the invention, the catheter section of the VAS is reinforced with polymeric filament, metallic wire or fibers, or combination thereof, and preferably in a spiral configuration. Reinforcement of the insertion segment of the VAS, especially with metallic wire or fibers, may be used to provide an insertion segment with a reduced outer diameter and one that has improved anti-kink and/or crush-resistant properties compared to a similar catheter section lacking reinforcement. The wire or line may be bonded to the outer or inner surface of the catheter section, or may be extruded with or molded into the silastic material to form the catheter section. In some embodiments, a spiral wire is placed or bonded to the outer surface of a conduit material and then spray or dip coated with a material to provide a smooth outer surface that is not interrupted by the wire reinforcement. One of skill in the art will understand that other reinforcement configurations besides a spiral configuration may be used, including discrete or interconnected rings, circumferential and/or longitudinal fibers that may be aligned, staggered or randomly positioned in or on the walls of the VAS.

In one example, the catheter section comprises a silicone extruded tube with a nylon winding for reinforcement. The silicone may contain from about 1% barium to about 30% barium to improve the radio-opacity of the catheter section. In other embodiments, the silicone may contain from about 5% to about 20% barium, and in still other embodiments, the silicone may contain from about 10% to about 15% barium. Other radio-opaque materials may be substituted for barium or used in addition to barium. The nylon winding may comprise a nylon monofilament with a diameter of about 0.005 inch diameter to about 0.050 inch diameter, and preferably about 0.010 inch to about 0.025 inch diameter. The winding may be configured for a wrap of about 10 to about 60 per inch, preferably about 20 to about 40 per inch. Silicone over molding, step up molding and/or silicone spray may also be used to provide a more consistent and/or smoother outer diameter over the portions of the catheter section.

In another example illustrated in FIGS. 6A and 6B, the catheter section 106 comprises a silicone tube 124 with Nitinol winding 126 for reinforcement. The Nitinol winding 126 may have a diameter of about 0.002 inch diameter to about 0.020 inch diameter, and preferably about 0.003 inch diameter to about 012 inch diameter. The Nitinol winding 126 may be configured for a wrap of about 10 to about 100 per inch, and preferably about 20 to about 60 per inch. The outer surface of the catheter section 106 is sprayed with silicone 128 to provide a more uniform and smoother outer diameter.

In one specific embodiment, the catheter section of the VAS comprises an insertion segment reinforced with spiral Nitinol wire, and a connecting segment reinforced with polymeric spiral filament. The insertion segment of the catheter section is adapted to be inserted into a vein while the connecting segment is adapted for attachment to a conduit connector and/or to the graft section of the VAS. By using metal wire for the insertion segment of the catheter section, smaller outer diameters may be achieved to facilitate insertion of the catheter section of the VAS through the skin and into a vein or other blood vessel. On the other hand, by providing polymeric reinforcement of the connecting segment, the diameter of the connecting segment may be reduced while maintaining the ability to trim the connecting segment of the catheter section without creating a sharp end or burr that may result when cutting through a metal wire reinforced portion of the catheter section. The insertion segment may have a length of about 10 cm to about 50 cm, preferably about 15 cm to about 35 cm, and most preferably about 20 cm to about 25 cm. The connecting segment of the catheter section can have a pre-trimmed length of about 10 cm to about 50 cm, preferably about 15 cm to about 35 cm, and most preferably about 20 cm to about 25 cm. In some embodiments of the invention, the total length of the catheter section is about 20 cm to about 250 cm, sometimes about 30 cm to about 60 cm, and other times about 120 cm to about 250 cm. Longer lengths may be used when implanting the device between axillary/femoral sites.

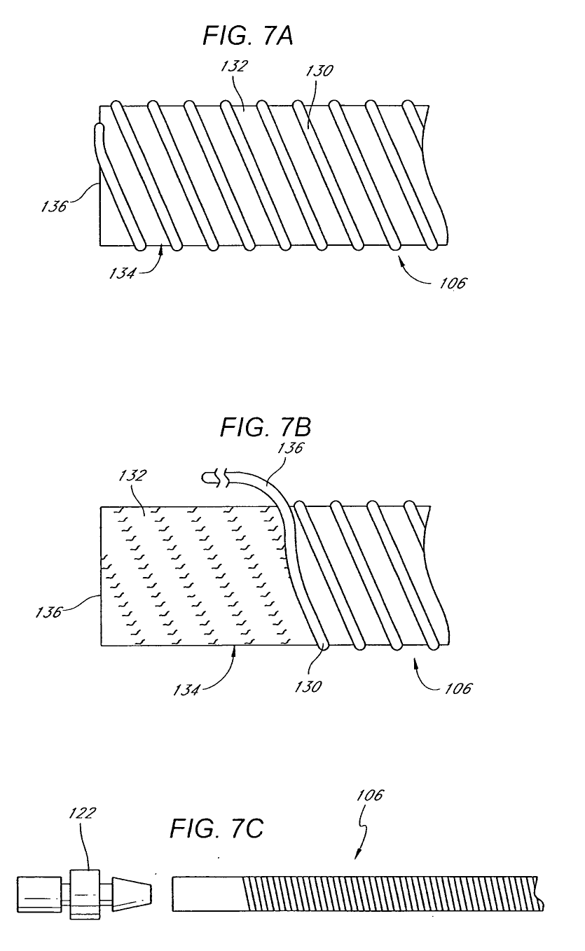

In further embodiments of the invention, depicted in FIG. 7A, the polymeric reinforcement 130 of the catheter section 106 is bonded or adhered to the outer surface 132 of the connecting segment 134, rather than embedded within the wall of the connecting segment 134. In some embodiments, such as those in FIGS. 7A and 7B, the polymeric reinforcement 130 is also bonded or adhered in a manner that allows the controlled peeling or separation of a portion of the polymeric reinforcement 130 from the outer surface 132 of the connecting segment 134, without damaging or violating the integrity of the remaining structure of the connecting segment 134. Referring to FIG. 7C, this feature may be beneficial in embodiments of the invention where the polymeric spiral reinforcement 134 resists or prevents the radial expansion of the connecting end 136 needed in order to fit the end of the connecting end 136 over a conduit connector 122. By allowing the controlled removal of a portion of the polymeric reinforcement 130, after trimming the connecting segment 134 of the catheter section 106 to its the desired length, a portion 136 of the polymeric reinforcement 130 may be removed from the connecting segment 124 in order to prepare the catheter section 106 for fitting to a conduit connector 122 or an integrated connector on a graft section of a VAS. In a similar fashion, the reinforcement may preferably be embedded in the catheter wall but close to the outer surface to enable easy removal.

To reduce the risk of damage to the catheter section and/or blood vessel structures where the catheter section is inserted, and/or to reduce the turbulent blood flow at the distal opening of the catheter section, the edge of the distal tip of the catheter section may be rounded. In some embodiments, rounding may be performed with a silicone dip or shadow spray, or may be molded to a round shape.

3. Implantation of the Vascular Access System

In some embodiments of the invention, the low profile of the VAS, combined with the ease of inserting the catheter section of the VAS into the vasculature, allows the use of a minimally invasive procedure to implant the device in the body. Depending upon the diameter of the catheter section of the VAS, the catheter section may be inserted into the vein using an open surgery technique, or preferably a venous cutdown, or most preferably by Seldinger technique. These techniques are well known procedures to those of ordinary skill in the art.

Once the insertion site of the catheter section of the VAS is established, a subcutaneous pathway from the catheter section insertion site to the desired graft section attachment site may be created using any of a variety of specialized tunneling instruments or other blunt dissection tools. The VAS system is then passed through the subcutaneous pathway and the graft section is attached to the desired site. A single, uninterrupted subcutaneous pathway may be created between the insertion site and attachment site of the VAS, particularly where the VAS device comprises a unibody design. Depending upon the sites selected, the particular anatomy of a patient, the tortuosity of the desired subcutaneous pathway, and/or the modularity of the VAS, it may be desirable to create one or more intermediate surface access sites along the subcutaneous pathway to make it easier to perform the subcutaneous tunneling and/or to pass one or more sections of the VAS along the pathway. The use of intermediate surface access sites is particularly desirable, but not necessary, when implanting a multi-section VAS. The individual sections of the VAS may be implanted separately along the sections of the subcutaneous pathway, and then attached via conduit connectors or other structures at the intermediate surface access points and then buried subcutaneously.

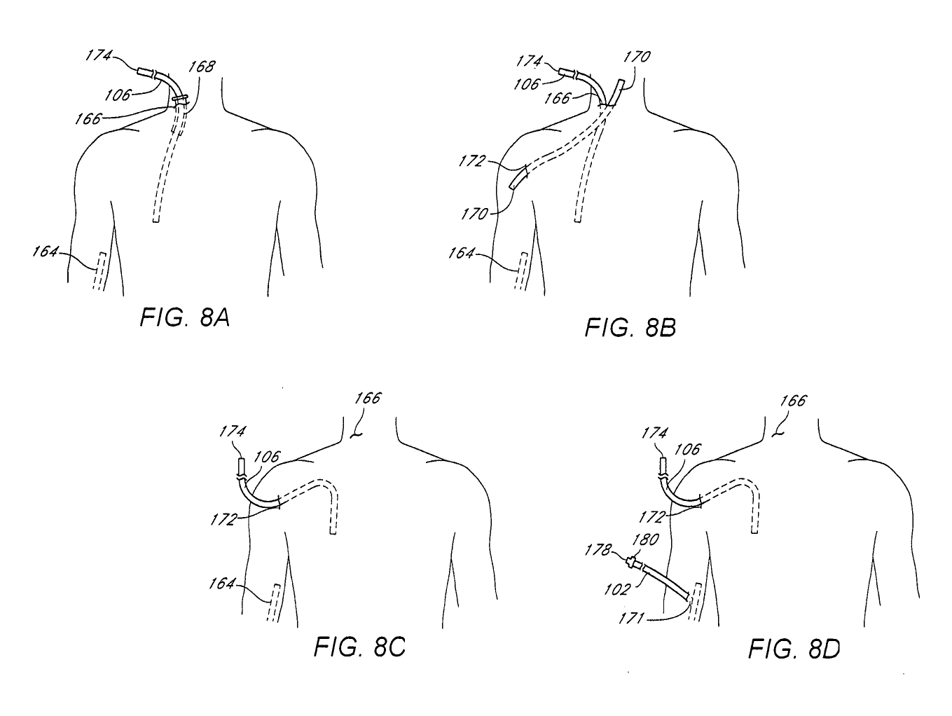

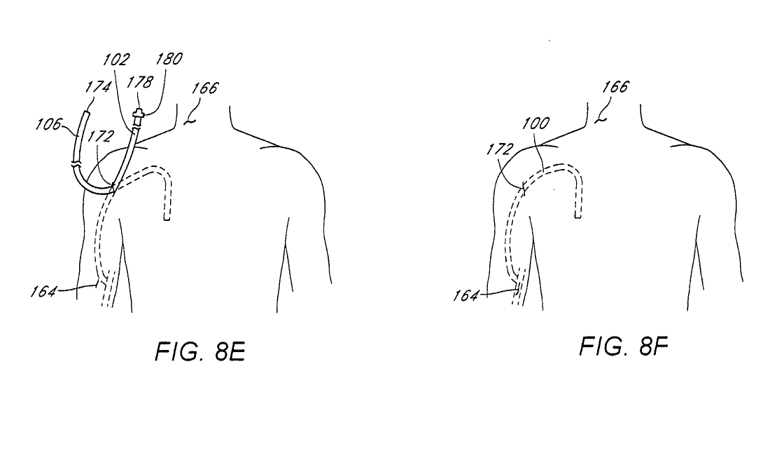

Referring to FIGS. 8A to 8F, in one embodiment of the invention, the patient is prepped and draped in the usual sterile fashion. Either local or general anesthesia is achieved. In FIG. 8A, the brachial artery is palpated on the patient and terminal access site 164 is marked. The internal jugular (IJ) vein is located and an initial access site 166 to the IJ vein is selected using anatomical landmarks and/or radiographic visualization such as ultrasound. A guidewire is passed into the IJ vein and then a dilator is passed over the guidewire to facilitate insertion of an introducer into the IJ vein. A small scalpel incision may be needed at the guidewire insertion site if the skin and/or subcutaneous tissue create excessive resistance to the insertion of the dilator. The dilator is removed and an introducer 168 is inserted over the guidewire and into the IJ vein. The introducer 168 may be a standard or custom type of introducer. The catheter section 106 of the VAS is then inserted into the introducer, through the IJ vein and into the superior vena cava or right atrium. The position of the distal tip of the catheter section 106 is confirmed radiographically and the patient is checked for accidental collapse of the lung due to improper insertion. The introducer 168 is then removed, either by pulling the introducer over the proximal end of the catheter section, if possible, or by peeling away the introducer if a peel-away introducer was provided.

In FIG. 8B, a surgical rod 170 is then inserted into the subcutaneous space through the initial access site. The rod 170 is used to subcutaneously tunnel toward the anterior shoulder. In other embodiments, the subcutaneous tunneling and implantation of the VAS section may occur generally simultaneously. Once the anterior shoulder is reached, a scalpel is used to create an intermediate access site 172 to the rod 170. In FIG. 8C, the rod 170 is removed from the initial access site 166 and then the proximal end 174 of the catheter section 106 is passed through the subcutaneous pathway to exit from the intermediate access site 172. The same surgical rod 170 or a different rod is then inserted into the intermediate access site 172 and used to subcutaneously tunnel distally down the arm until the marked brachial artery site is reached. A terminal access site 164 to the rod is created and further exposed to access the brachial artery. The anastomosis end 171 of the graft section 102 of the VAS is attached to the brachial artery, as illustrated in FIG. 8D. Alternatively, the anastomosis may be performed after the graft section 102 is subcutaneously positioned. Referring next to FIG. 8E, the connector end 178 of the graft section 102, with pre-attached conduit connector 180, is passed from the terminal access site 164 to the intermediate access site 172. A connector sleeve with integrated strain relief structure may be passed over the proximal end 170 of the catheter section 172. The initial and terminal access sites 166, 164 are checked for any redundant conduit and pulled taut from the intermediate access site 172 if needed. The proximal end 174 of the catheter section 106 is trimmed to the desired length. About 0.5 cm to about 1 cm segment of nylon winding at the trimmed end of the catheter section is separated and cut away. The proximal end 174 of the catheter section 106 is fitted to the pre-attached conduit connector 180 of the graft section 102. The catheter section 106 is secured to the conduit connector 180 with a crimp ring and the connector sleeve is repositioned over the conduit connector. The exposed portions of the conduit connector 180, attached to the distal end 178 of the graft section 102 and the proximal end 174 of the catheter section 106, are either pulled from the graft end or pushed into the subcutaneous space through the intermediate access point 172, as illustrated in FIG. 8F. Flow through the VAS 100 is reconfirmed either by palpation or preferably by ultrasound and/or angiography. The three access sites 164, 166, 172 are sutured closed. The implanted VAS 100 is then accessed with hemodialysis needles to perform hemodialysis.

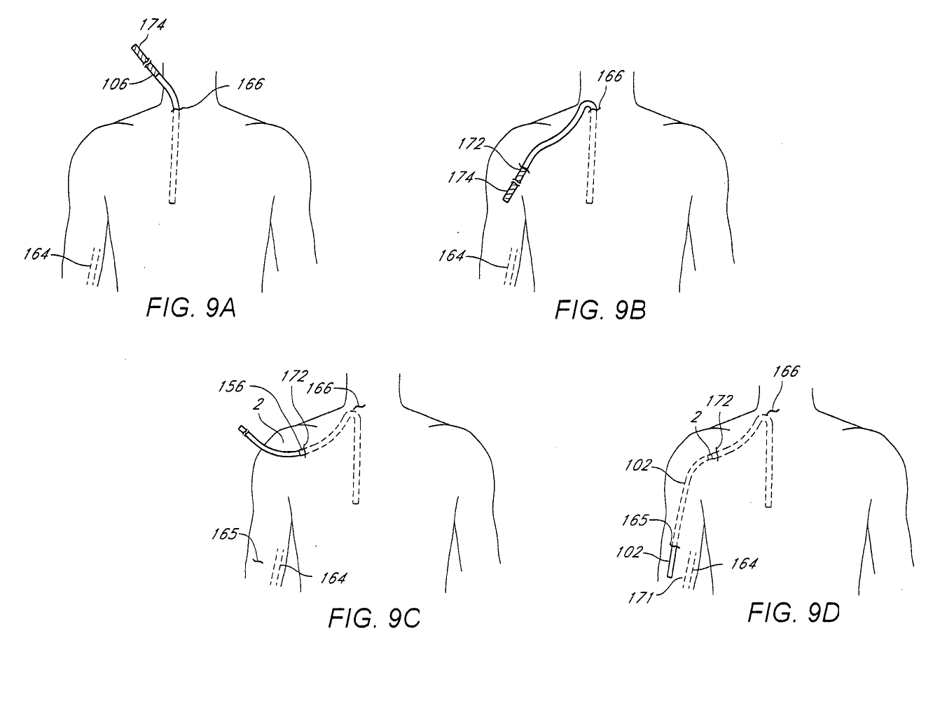

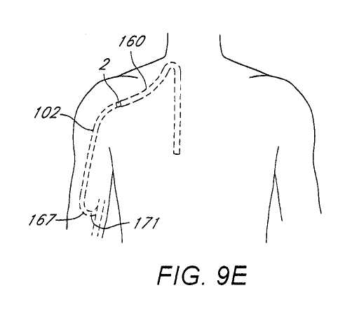

In a preferred embodiment of the invention, depicted in FIGS. 9A to 9E, the patient is placed under general anesthesia and the graft routing is marked on patient arm. The surgical site prepped, sterilized and draped. An incision 166 is made in the neck to access the lower portion of internal jugular vein. A small wire is inserted through the access site 166. The small wire is exchanged with a mid-sized introducer set (about 5 F to about 14 F) and the wire is removed. The vein may be angiographically assessed, and if a stenosis is identified that may preclude advancement of catheter, angioplasty may be used to enlarge the lumen of the vein. A larger wire is inserted through mid-sized introducer. The mid-sized introducer is exchanged with 20F introducer. The patient is preferably placed in Trendelenberg position prior to the removal of the dilator to reduce the propensity for air introduction upon catheter insertion. The dilator and clamp introducer is removed and the introducer is closed off with a finger. The catheter 106 is filled with heparinzed saline, clamped and inserted through the introducer. The ventilator may be optionally turned off while catheter is inserted to reduce the propensity for introduction of air. The introducer is peeled away, leaving the catheter 106 in the IJ, as shown in FIG. 9A. A “Christmas Tree” valve or atraumatic clamp (preferably a Fogarty's clamp) may be used to stop back bleed through catheter. The patient may be brought out of Trendelenberg position. The position of the catheter tip is checked under fluoroscopy for a position in the proximal to mid-right atrium (RA), and is adjusted if needed. To tunnel the catheter subcutaneously, a delta-pectoral incision 172 is made, as shown in FIG. 9B. The catheter 106 is then tunneled to the delta-pectoral incision 172 by routing above the sternocleidomastoid muscle in a sweeping fashion. Depending upon the characteristics of the catheter 106, in some instances care should be taken to not create a bend in the catheter 106 with a diameter less than about 2.5 cm to avoid kinking. The nylon filament on the catheter 106 is wound down and the catheter 106 is cut to leave approximately an inch outside of delta-pectoral incision 172. An appropriate amount of nylon winding is removed in comparison to the length of the barb on the connector 2. A connector sleeve 156 (flower end first) and crimp ring are placed over the catheter, typically in that order, depending upon the particular securing mechanism used. As depicted in FIG. 9C, the connector 2, pre-attached to the graft 102, is then attached to the catheter 106, and the catheter 106 is secured to the connector 2 using the crimp ring. The connection is tested to ensure integrity. The connector sleeve is 156 placed over most if not all the exposed metal surfaces. A brachial incision 164 is made to expose the brachial artery. An auxiliary incision site 165 is made lateral to the brachial incision site 164. The graft 102 is tunneled from the delta-pectoral site 172 or connector incision site in a lateral-inferior direction until reaching the lateral aspect of the arm. It is preferable but not required to stay superficial and also lateral to the bicep muscle. Tunneling is continued inferiorly until the auxiliary incision site 165 is reached. A tunnel from the auxiliary site 165 to the brachial site 164 is then performed to create a short upper arm loop in a “J” configuration 167 just proximal to the elbow. The graft is then tunneled cephalad along the medial aspect of the upper arm to the brachial incision site 164. Preferably, the graft 102 should be parallel to the brachial artery to allow construction of a spatulated anastomosis. The orientation line or marks are checked for an orientation in the same direction at both ends 171, 178 of the graft 102 and to verify that the catheter 106 has not moved from the proximal RA. The graft 102 is checked for a sufficient amount of slack. A parallel end-to-side anastomosis is then constructed by cutting the graft at an oblique angle and making an arteriotomy along the long axis of the brachial artery. This may be advantageous as it may cause less turbulence at the anastomotic site and may be less prone to stressing the anastomosis. The anastomosis between the artery and graft is then performed as known to those of ordinary skill in the art, as shown in FIG. 9E. A Doppler scan of the lower right arm and hand may be performed prior to closing to check whether steal syndrome occurs with the shunt. The anastomosis is checked angiographically via back-filling along the length of the VAS. Tip placement in the RA and VAS integrity with movement of the subject's arm may also be checked. Patency and absence of significant bends or kinks is also checked. The incisions are closed and dressed.

Although the embodiment described above utilizes the internal jugular vein and the brachial artery as the insertion and attachment sites, respectively, of the graft system, one with skill in the art will understand that other insertion and attachment sites may be used, and were described previously above. For example, other arteries that may be used with the invention include but are not limited to the ulnar artery, radial artery, femoral artery, tibial artery, aorta, axillary artery and subclavian artery. Other venous attachments sites may be located at the cephalic vein, basilic vein, median cubital vein, axillary vein, subclavian vein, external jugular vein, femoral vein, saphenous vein, inferior vena cava, and the superior vena cava. It is also contemplated the implantation of the device may be varied to configure the graft system in a generally linear configuration or a loop configuration, and that the insertion and attachment sites of the invention need not be in close proximity on the body. For example, attachment and insertion of the device may be performed at an axillary artery and femoral vein, respectively, or from a femoral artery to an axillary vein, respectively.

B. Instant Access

In some embodiments of the invention, the VAS is configured to provide immediate hemodialysis access upon implantation, while reducing or eliminating the risk of hemorrhage associated with accessing the graft section of the VAS prior to its maturation or without inserting an additional catheter to provide temporary dialysis access. The instant access sites may be provided as subcutaneous needle access sites that use self-sealing materials or other structures to stop the bleeding once the hemodialysis needles are removed. The instant access sites may also comprise temporary catheters attached to VAS that exit the skin to provide external access to the VAS with a further benefit of eliminating the discomfort associated with piercing the skin to achieve hemodialysis access. These and other embodiments of the invention are discussed in further detail below. These embodiments may be well suited for integration into medical devices other than VAS, including but not limited to any of a variety of traditional dialysis graft designs, access graft designs, catheters, needle access ports or intravenous fluid tubing.

1. Instant Access Materials

In one embodiment of the invention, the graft or catheter material may have self-sealing properties. Self-sealing refers generally to at least at portion of the VAS wall having the ability to reseal following puncture with a sharp instrument, such as a needle. A material with self-sealing properties may be used immediately upon implantation, in contrast to traditional graft materials. No biological maturation process to improve the leakage properties of the material is required. A self-sealing material may also reduce the time required to stop bleeding from the access site following removal of the hemodialysis needles. Furthermore, the material may also be used to provide instant access sites at other sections of the VAS, or in other medical products which may benefit from self-sealing properties. The instant access material may be located anywhere along the VAS. In one embodiment of the invention, a low durometer material may be used as an instant access site. In one embodiment of the invention, low durometer materials comprise materials having a hardness of about 10 to about 30 on the Shore A scale, and preferably about 10 to about 20 on the Shore A scale. Other structures with self-sealing properties are described below.

a. Residual Compressive Stress

In another embodiment, the invention provides a graft or catheter comprising a conduit having residual compressive stress to provide self-sealing properties to the graft or catheter. In one embodiment, the self-sealing conduit material is constructed by spraying a polymer, preferably a silicone, onto a pre-existing tube of conduit material while the tube is subject to strain in one or more directions. The self-sealing material provides mechanical sealing properties in addition to or in lieu of platelet coagulation to seal itself. In one embodiment, the VAS comprises a self-sealing material having two or more alternating layers of residual stress coating.

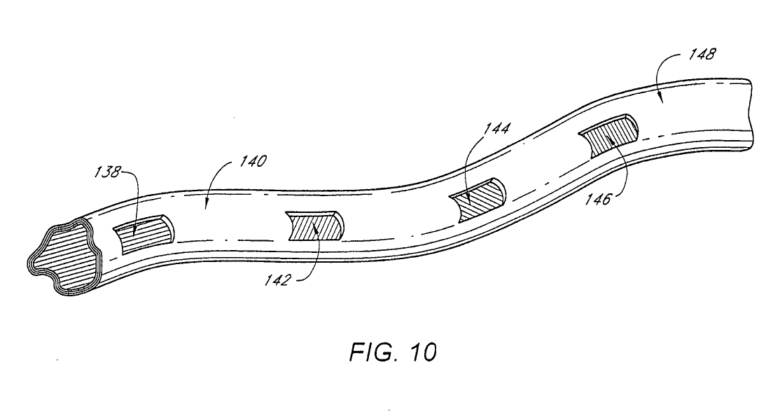

In one particular embodiment, illustrated in FIG. 10, the conduit material comprises four layers, wherein the inner layer 138 is formed by axially stretching the conduit material 140, spray coating the conduit material and allowing the coating to cure, then releasing the conduit material from tension. The second layer 142 (from inner layer) is formed by twisting the conduit material 142 about its axis, spray coating and curing it, then releasing it from torque. The third layer 144 is formed by taking the conduit material from the previous step and twisting it about its axis in the opposite direction of previous step, spray coating and curing it, then releasing it from torque. The fourth layer 146 is created by taking the product from previous step, expanding it with internal pressure, spray coating and curing it, then relieving the material of pressure. Note that this may also create an axial strain since the tube elongates with pressure. A fifth optional layer 148 of an additional strain coating or a neutral coating may also be provided. The additional layer 148 may aid in achieving consistent outer diameter.

Although examples are provided above for creating a self-sealing graft or catheter material, one of ordinary skill in the art will understand that many variations of the above processes may be used to create a self-sealing conduit material. One variation is to produce residual stress in the graft material by inflating and stretching the material to a thin wall and applying polymer to the wall either by dipping or spraying. The amount of circumferential and/or axial stress in the final tube may be controlled separately by adjusting the amount of inflation or axial stretch. Also, the above steps may be performed in a different order, and/or or one or more steps may be repeated or eliminated. Other variations include spraying a mandrel without using a pre-existing tube or turning the conduit material inside out (for compressive hoop stress) for one or more steps.

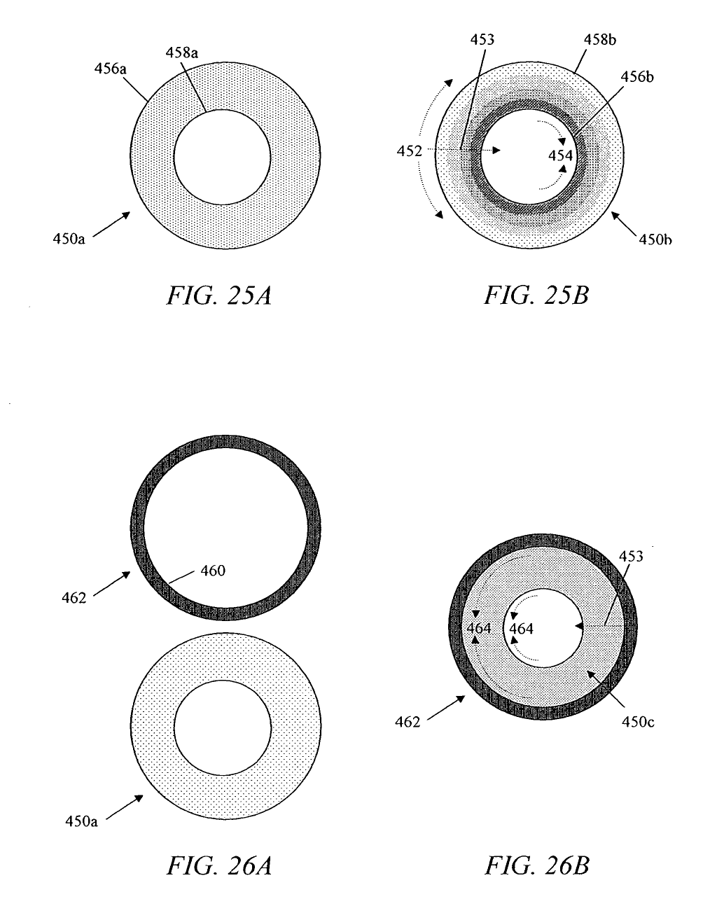

In another embodiment, residual compressive stress may be provided by using a silicone tube that is turned inside out. Turning the silicone tube inside out, i.e. everting the tube, results in stresses and strains that create highly compressed silicone about the inner lumen of everted tubing. Referring to FIGS. 25A and 25B, eversion of a silicone tube 450a creates a circumferential tension 452 and circumferential compression 454 in the everted tube 450b. By everting the silicone tube 450a, the pre-everted outer surface 456a has been elastically compressed to form the inner lumen 456b of the everted tube 450b, and the pre-everted inner surface 458a has been elastically expanded with a tension force 452 the outer surface 458b of the everted tube 450b. The tension force 452 on the outer portions of the everted tube 450b which causes a radial compression force 454 about the inner surface 456b of the everted tube 450b. The tension force 452 may also exert a radially inward force 453 on about the inner surface 456b of the everted tube 450b. These forces thus act to increasingly compress the self-sealing material along a radially inward increasing vector.

Unlike multi-layer self-sealing structures which often have discrete compressive forces at leach layer, an everted tube 450b will have a gradual or continuous change in compressive force along the radius of the tube 450b. In some instances, the everted tube 450b may be characterized as having an intermediate radius or depth where the outer tension force 452 is canceled by the inner compressive force 454. Put another way, the everted tube smoothly transitions from an outer zone of less dense elastomeric material to an inner zone of elastomeric material having greater density, with an intermediate zone between the outer and inner zones that has a density about equal to the pre-everted density of the elastomeric tube.

Although the silicone tube 450a depicted in FIG. 25A has a uniform density and structure, in other embodiments, the silicone tube 450a may have variable density and/or geometry along one or more dimensions of the silicone tube 450a. Thus, the silicone tube 450a may have a variable density or structure radially, circumferentially, longitudinally or in any combination thereof, including helical variations. In still other embodiments, elastomeric structures having existing self-sealing properties may be further enhanced by eversion.

It is understood that eversion may or may not change the internal and/or outer diameter of the elastomeric tube. Likewise, eversion may or may not alter the length of the tube from its pre-everted length. The degree of change, if any, may depend on material properties of the elastomer, as well as any other materials that may be coupled or bonded to the elastomer. In some embodiments, the circumferential tension and compression forces will largely cancel each other, resulting in little diameter change of the elastomeric tube. Likewise, in most embodiments of the invention, little if any change in length will be observed post-inversion.

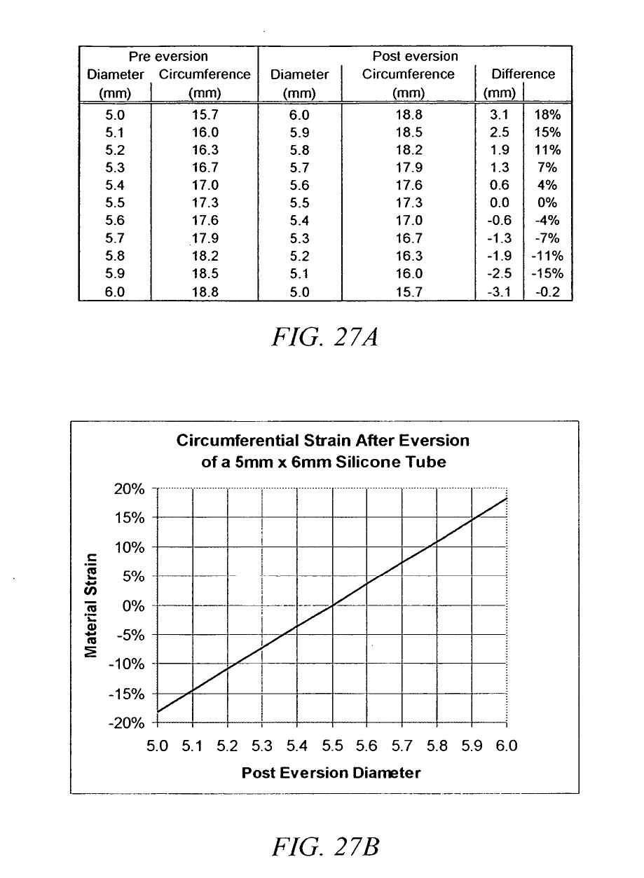

In one specific example, a 50 durometer silicone tube with a 0.197″ (5 mm) ID and a 0.236″ (6 mm) OD and a length of 50 mm was everted. The post-eversion length was unchanged while the post-eversion diameters were 0.202″ ID and 0.240″ OD. As the measurement tolerance for the ID is about 0.001 to 0.003″ and the measurement tolerance for the OD is about 0.001″ to 0.002″, there was no significant change in the post-eversion dimensions of the silicone tube. These empirical findings comport with the predicted changes in a 5 mm ID×6 m OD silicone tube. FIG. 27A is a table listing the predicted strain in an everted silicone and FIG. 27B graphically illustrates the predicted circumferential strain in the everted tube.

While eversion reduces leakage of the silicone tube following needle puncture, large strains from bending or pulling on the silicone tube may still result in significant leakage of the everted tube. This may occur when a silicone tube is bent or stretched, for example where the silicone material along the greater curvature of a bend stretches and opens up needle puncture holes, resulting in leakage.

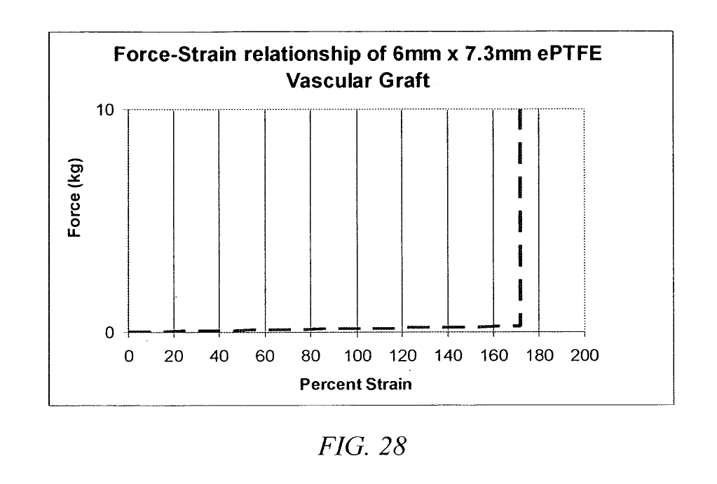

To resist the effects of these larger strains that may cause leakage, such as from bending or pulling, a leak-resistant material such as silicone may be reinforced with expanded polytetrafluoroethylene (ePTFE). ePTFE has a property related to its longitudinal bias, in that in its resting state it has a relatively limited axial stretch property, while still axially compressible to a larger degree. FIG. 28 graphically depicts the stretch-resistant properties of ePTFE. Data in the graph was generated using a 6 mm×7.3 mm ePTFE vascular graft. As the graft is stretched up to about 170%, only a small amount of stretch-resistant force is generated by the ePTFE. Once the ePTFE is stretch beyond about 170%, however, the stretch-resistant begins to increase substantially. To utilize this property of ePTFE, an overlay of ePTFE may be placed, for example, over the silicone tube to resist stretching of the silicone. The tube can still bend freely (although sometimes slightly less than a tube without an overlay of stretch-resistant material) since the lesser curvatures of the bend undergoes compression while the outside does not stretch. If the ePTFE were not present, the outer curvature would stretch while the inner curvature experienced compression. Typically, the ePTFE is stretched to an internodal spacing of about 25 microns to about 30 microns for use in reinforcing a silicone layer. In other embodiments, the ePTFE may be stretched to an average internodal spacing of about 20 microns to about 35 microns, and sometimes to an average internodal spacing of about 20 to about 40 microns.

Referring to FIGS. 26A and 26B, circumferential compressive forces may also be formed in an elastomeric structure, for example, by radially compressing a silicone tube 450a. In one example, a silicone tube 450a is compressed by inserting it into the inner lumen 460 of a smaller compression tube 462, or by coupling to some other circumferentially compressive structure. When the larger silicone tube 450a is forced into the smaller compression tube 462, the silicone tube 450a is compressed into a compressed silicone tube 450c, which increases the seal-sealing properties of the tube 450c. The magnitude of the forces will vary depending upon the degree of radial compression. In some of these embodiments, the circumferential compressive forces 464 along the radial depth of the silicone tube 450c may be more evenly distributed compared to an everted silicone tube 450b, but it may be more difficult to achieve the magnitude of compression about the inner surface of the everted silicone tube. A radially inward force 453 may also be exerted by the smaller compression tube 462 onto the compressed silicone tube 450c. Unlike the everted silicone tube, however, the inner lumen of the radially compressed silicone tube will typically be smaller, depending on the degree of radial compression.

In addition to using a stretch-resistant material such as ePTFE to limit stretching of the everted silicone tube, the silicone tube may also be placed under varying degrees of longitudinal compression prior to bonding to the stretch-resistant material. This may further improve the leak-resistant properties of the everted self-sealing material by placing under longitudinal compression. In some embodiments, a longitudinal compressive strain of about 1% to about 10% may be applied to the silicone tubing during bonding, with strains in the range of about 3% to about 4% being preferred. Although strains greater than 10% may be used, silicone tubing may start buckling at larger strains and become more difficult to manufacture. The formation of longitudinal compressive forces in the silicone tube may results in expansion of the outer diameter of the tube and reduction in the inner diameter. In some embodiments, the ePTFE material may be coupled to the self-sealing elastomeric material prior to undergoing eversion.

Although other combinations of materials may also be used to provide stretch-resistance to a leak-resistant material, ePTFE has a long history of use in vascular applications. ePTFE allows cellular growth into outside surface, which reduces the likelihood of infection and improved device stability. Silicone and ePTFE also have repeatable performance and degrade minimally over time. In other embodiments, another biocompatible, but not necessarily a hemocompatible, material that exhibits a higher resistance to stretching than compression may be used in lieu or in addition to ePTFE. Similarly, another material with mechanical properties similar to silicone, possibly polyurethane, may be used in place of silicone. Thus, the examples described above are embodiments of a broader concept of an implant having a leak-resistant layer with a stretch-resistant structure to limit overstretching of the leak-resistant layer. The stretch-resistant structure may be a stretch-resistant layer bonded to the leak-resistant layer, or a stretch-resistant structure embedded within the leak-resistant layer. Preferably, the leak-resistant layer comprises silicone or the walls of a silicone tube. Preferably, the stretch-resistant structure is a stretch-resistant layer, and most preferably a layer of ePTFE. Other suitable biocompatible, but not necessarily hemocompatible, material that exhibits a higher resistance to extension than compression may be used in conjunction or in lieu of ePTFE or PTFE.

Although many types of silicone may exhibit the properties described above, medical grade silicone polymers with suitable biocompatibility and stability are preferred. Cross-linked, heat cured and/or room temperature vulcanizing (RTV) moisture cured silicones may be used. In preferred embodiments, a low durometer (about 5 to about 50 on a Shore A scale), flexible, high tear strength silicone are used because such silicones will conform to an inserted needle more easily. One of ordinary skill in the art will also understand that other materials having elastomeric properties may also be used to form self-sealing materials by eversion. These other materials include polyurethanes, preferably also those with a low durometer.

In one embodiment, a method of manufacturing an instant access material is provided. A silicone tube is placed on a mandrel and sprayed with one or more layers of silicone. The silicone tube is axially rolled back to assume an inside-out configuration. A Nitinol winding is applied to the tube in order to provide kink resistance to the tube and the winding is coated with one or more layers of silicone. A portion of ePTFE graft tubing is expanded with a tapered mandrel. Other winding or threading, such as nylon or stainless steel may also be used. The ePTFE graft tubing typically has an inner diameter equal to or slightly larger than the inner diameter of the silicone tube. The graft may have a constant inner diameter (ID) and/or outer diameter (OD), or may have a slight change or tapering in ID and/or OD. In one example, a silicone tube with a 5 mm inner diameter and 1.25 mm wall thickness is used with a standard 6 mm ID ePTFE vascular graft material. The expanded graft material is placed over the aforementioned silicone tube and the ePTFE graft material is bonded at one end to the silicone tube using an adhesive, such as a silicone adhesive. The remaining portions of the silicone tube are lightly compressed as the remaining ePTFE graft material is put under tension and the remaining end of the ePTFE graft material is bonded in place with adhesive. Typically, the tension force exerted on the ePTFE is the equal and opposite to the compression force acting on the silicone, but in other embodiments, the magnitude of the force or forces may be different.

In one specific embodiment of the invention, a silicone tube was inverted and sprayed with silicone to an average outer diameter of about 0.24 inches to about 0.30 inches, and preferably about 0.25 inches to about 0.29 inches, and most preferably about 0.25 inches to about 0.29 inches. Preferably, a two-part silicone (e.g. Nusil MED 6233) diluted with xylene to a 40% silicone may be used as a spray, but one of ordinary skill in the art will understand that a variety of silicone or non-silicone spray materials having generally similar characteristics may also be used. An ePTFE graft (Boston Scientific Exxcel) was stretched over a 22 French Cook C-PLI-22-38 Peel-Away dilator and placed over the silicone tube catheter. The graft was bonded with a silicone adhesive at its proximal end to the catheter and allowed to cure. The graft was then held taut or placed under light tension while the catheter was held at relaxed length or under slight compression. The distal end of the graft was then held to the catheter with a circumferential wire twist tie approximately 0.25″ from the graft end for temporary clamping. The protruding portion of graft material was then bonded with silicone adhesive. The device was cured in an oven at about 125 degrees Celsius for about 10 minutes before the wire twist tie was removed. The device was leak-tested with water at about 127 cm H₂O. A 17 gauge needle was inserted at an angle into the device three times with no leakage observed during the insertion or after removal of the needle.

In alternative embodiments of the above device and process, the ePTFE may be bonded to the inside of the silicone tube or embedded within the silicone tube. The silicone tube need not be pre-formed, e.g., it may be formed simultaneously by spraying a bare mandrel or ePTFE directly. Other methods of silicone application, such as dipping and injection molding may also be used at any time in place of spraying. The ePTFE may also vary in size and also be placed over the silicone tube without expansion, for example, by using a lubricating agent and/or by shrinking the silicone tube with vacuum pressure. The silicone material need not be in a tubular form and may or may not have an inherent residual compressive stress, as the compression may be provided once the ePTFE material is prepared and bonded to the silicone material. Likewise, the ePTFE material need not be in the form of a preformed graft tube. The ePTFE may be provided in strips that are wrapped or bonded to the silicone tube. The ePTFE may be spray coated with silicone and possibly turned inside out; or turned inside out, spray coated with silicone, and turned inside out again. The tube may also be reinforced with a winding made from nitinol, nylon or stainless steel, for example.

In another embodiment, stretch or elongation of the leak-resistant material is controlled by embedding flexible fibers or strands of material along the length of the leak-resistant material. In some embodiments, the fibers or strands may be oriented along a particular axis of the leak-resistant material. In other embodiments, the fibers or strands may not have any particular orientation, but become more longitudinally oriented as the leak-resistant material is stretched. The fibers, strands or other elongate structural members may comprise nylon or other similar material that does not have significant stretch or elongation properties but exhibits greater compressive properties. These compressive properties allow the leak-resistant material to maintain its flexibility while still resisting stretch or elongation. In some embodiments, the increased compression may be the result of the thin fibers buckling under compression. In other embodiments, the fibers may or may not be embedded directly into the self-sealing layer, but are part of the inner or outer surface of the self-sealing layer, or are embedded in a secondary layer joined or bonded to the self-sealing layer. In still other embodiments where the fibers are embedded into the self-sealing layer, a single-layer self-sealing graft may be used because the self-sealing layer will have the properties of a stretch resistant layer without requiring a second layer.

b. Open, Porous Structure

In another embodiment of the invention, a self-sealing portion of the VAS comprises a porous structure (e.g. material similar to Perma-Seal by Possis Medical or Vectra by Thoratec) in the wall of the VAS catheter or graft. Resistance to blood leakage in this device results from a porous wall design that provides increased surface area to promote blood clotting. In addition, the porous design can recover more readily after a needle has been left in the wall for several hours. The outer surface of the catheter is preferably porous to facilitate in-growth of tissue in order to further facilitate sealing and, more importantly, to minimize the likelihood of infection.

c. Intrawall Gel

In another embodiment of the invention, the self-sealing material comprises one or more soft inner gel layers within a wall region of the VAS. The wall region and gel layers are pierceable by a needle. As the needle is removed, the gel seals the needle tract because the gel is flexible and semi-gelatinous. A whole range of materials could be used; one specific embodiment is described in U.S. Pat. No. 5,904,967 to Ezaki; another material classification is organosiloxane polymers having the composition of:

65%-Dimethyl Siloxane

17%-Silica

9%-Thixotrol ST

4%-Polydimethylsiloxane

1%-Decamethyl cyclopentasiloxane

1%-Glycerine

1%-Titanium Dioxide

d. Instant-Access Graft Devices

As mentioned previously, the instant access materials disclosed herein may be used with the preferred embodiments of the invention comprising a graft component and a catheter component, but can also be incorporated into more traditional vascular access graft designs.

For example, the instant-access materials may be bonded to a traditional tubular vascular access graft comprising ePTFE. In addition to providing instant-access properties, the instant access region may also provide faster or instant hemostasis. This can aid in performing dialysis because it reduces bleeding through the graft. Reduced bleeding may result in reduced pain, swelling, infection rate, and bleeding complications such as hematoma. Bleeding may be reduced when the needles are removed or if the graft is accidentally “backwalled” (sticking the needle all the way through the graft). Backwalling the graft is a significant concern with standard grafts because, in order to stop the bleeding, a substantial pressure must be applied to the graft in order to stop the bleeding at the inner wall. This pressure can occlude the graft, necessitating a thrombectomy or other declotting procedure to restore flow. The instant access region may also be more resistant to collapse or compression. This can aid in the localizing the instant access region and aid the insertion of dialysis needles. The instant-access material(s) may be provided along the entire length and circumference the ePTFE graft or to a limited section or sections of the ePTFE graft. The instant-access material may be bonded to the interior surface and/or exterior surface of the graft as well as between layers of ePTFE comprising the graft. The use of the instant access-material with ePTFE provides the sealing properties of the instant-access material along with an ePTFE sections that clinicians are familiar with and have traditionally used.

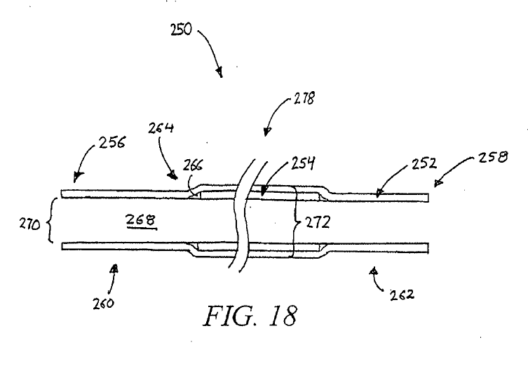

Referring to FIG. 18, in one embodiment of the invention, the instant-access graft 250 comprises a length of ePTFE tubing 252 with a smaller length of instant-access material 254 formed or bonded to the interior lumen. The instant-access material 254 is bonded between the two ends 256, 258 of the ePTFE tubing 252 such that the two ends 256, 258 of the ePTFE tubing 252 each end have a section 260, 262 lacking the instant-access material 254. A bare ePTFE section 260, 262 may be preferred for suturing to arteries, veins and other body conduits because of its similar compliance to vascular tissue and its suture retention strength. The ePTFE sections 260, 262 may also be preferred because it facilitates tissue ingrowth, which increases blood sealing capability and resistance to infection. To reduce the risk of thrombosis caused by turbulence at the interface 264 between the instant access material 254 and ePTFE tubing 252, silicone 266 or another bio-material may be used to fill in the interface gap 264 and to provide a smoother inner surface for the graft lumen 268. In some embodiments, to reduce or minimize changes to the inner diameter of the graft lumen 268, the ePTFE tubing may be pre-expanded to a larger diameter at the instant access site 278 in order to accommodate the volume of instant-access material 254, and thereby reduce or eliminate the intrusion of the instant-access material 254 into the graft lumen 268. Alternatively, the ePTFE and instant-access material may be expanded after bonding, but this may impair the function of the instant-access material.

By leaving the ends of the ePTFE graft 250 free of instant-access material 254, anastomoses of the two ends 256, 258 of the graft 250 to an artery and a vein remain similar to the anastomosis of traditional ePTFE-only vascular access grafts and therefore does not require further development of motor skills to implant the instant-access graft 250. In contrast, embodiments where the instant-access materials is provided at the ends of the ePTFE graft, the increased thickness of the combined ePTFE and instant access material may be more challenging for a surgeon to attach, and may cause increased thrombosis at the anastomotic sites due to differences in compliance with the blood vessel or due to lower quality surgical technique.

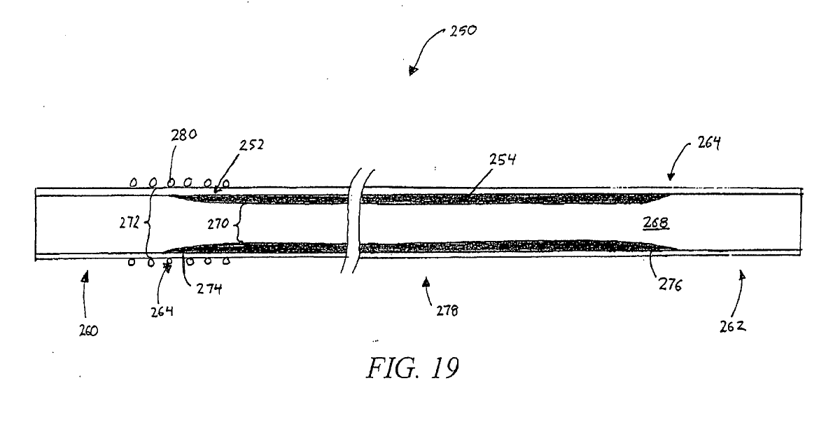

Although the embodiment depicted in FIG. 18 is configured to reduce or minimize changes to the inner diameter 270 of the vascular access graft 250, the outer diameter 272 of the graft 250 is increased in order to preserve the continuity of lumen 268. In other embodiments, as illustrated in FIG. 19, changes to the outer diameter 272 of the vascular access graft 250 may be reduced or minimized by providing instant-access material 254 along an lumen 268 of the ePTFE tubing 252, such that the instant-access material 254 displaces a portion of the lumen volume and results in a reduction of inner diameter 270. Turbulence at the interface 264 between the instant-access material 254 and ePTFE tubing 252 may be reduced by tapering the thickness of the ends 274, 276 of the instant-access material 254. One of skill in the art will understand that the relationship between the ID 270 and OD 272 of a graft 250 at the instant-access site may be adjusted accordingly. Furthermore, changes to both the inner and outer diameter 270, 272 of the graft 250 may be reduced by providing an ePTFE tubing 252 having a reduced thickness about the instant-access site 278 to compensate for its increased thickness due to the instant-access material 254.

FIG. 19 also depicts optional kink-resistance structure(s) 280 provided about the one or more ends 274, 276 of the instant-access material 254 that may resist kinking of the graft 250. A propensity to kink may result from differences in wall thickness and/or wall compliance between the instant-access material 254 bonded portion(s) 278 of the ePTFE tubing 252 and ePTFE-only sections 260, 262 of the graft 250.

In one specific embodiment of the invention, a two-layer self-sealing graft is provided. The graft comprises an outer layer of ePTFE graft material and an inner-layer of everted silicone tubing. Typically, the ePTFE has an ID that is larger than the ID of the everted silicone tubing. The ePTFE graft is slid over the everted silicone tubing and bonded with silicone adhesive. Preferably, the everted silicone tubing has a shorter length than the ePTFE graft material such that one or more ends of the device comprise ePTFE and not silicone. The transition from the inner diameter of the ePTFE graft to the everted silicone tubing may be molded with additional silicone to provide a smoother transition the two components. The portion of the outer ePTFE layer about the everted silicone tubing may also be radially expanded before and/or after bonding with the everted silicone tubing. The radial expansion may reduce the abrupt change, if any, in the inner diameter of the device at the transition and provide a more uniform inner diameter along the length of the device.

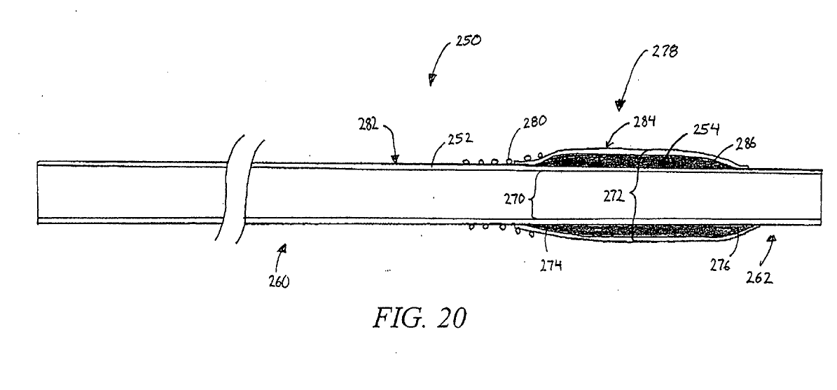

In a two-layer design, the ePTFE graft typically has an ID larger than the ID of the everted silicone tubing. For example, the silicone tubing may have a 5 mm ID (pre-eversion) and the ePTFE graft may have a 6 mm ID. The difference in ID may range from about 1 mm to about 3 mm, and preferably about 1 mm to about 2 mm. The everted silicone tubing may have an ID in the range of about 4 mm to about 10 mm, and preferably about 5 mm to about 8 mm, and most preferably about 5 mm to about 7 mm. One or both ends of the device preferably have segment lengths of about 0.25 cm or more ePTFE without self-sealing material, more preferably 1.5 cm or more and most preferably 3 cm or more. The silicone tubing may have a length of about 5 to about 80 cm, preferably about 8 to about 25 cm, and most preferably about 10 to about 17 cm. The graft material may have a length of about 10 to about 100 cm, preferably about 20 to about 50 cm, and most preferably about 30 to about 40 cm. FIG. 20 illustrates another embodiment of the invention whereby the inner diameter 270 of the graft 250 is generally preserved while providing an instant-access region 278. In this embodiment, the instant-access material 254 is bonded to the exterior surface 282 of the ePTFE tubing 252. Another piece or layer of ePTFE tubing 284 may be optionally overlayed on the exterior surface 286 of the instant-access material 254. By overlaying a second layer of ePTFE over the instant-access material, the patient's body is in contact only with the ePTFE and not the instant-access material, which may have a less favorable biocompatibility profile in some embodiments in comparison the ePTFE. Optional anti-kink structures 280 may also be provided about the ends 274, 276 of the instant-access material 254.

FIG. 20 also illustrates that the relationship between the length of the instant-access material 254 and the ePTFE tubing 252 may vary substantially. For example, a longer section 260 of ePTFE tubing 252 without instant access material 254 may be used to provide more traditional vascular access requiring endothelialization of the graft 250. By reducing the size of the instant-access section 278 relative to the overall length of the graft 250, the bulk of the graft 250, ease of implantation, cost of manufacturing, and/or manufacturing defect rate of the graft may be reduced while still providing sufficient instant-access function until the more traditional access becomes available in the ePTFE-only section(s) 260, 262. The silicone or instant access region is provided between the ePTFE end sections 260, 262 and its length may be varied depending on where the graft 250 is placed in the body. In some embodiments of the invention, the silicone section 278 has a net length of about 5 cm to about 40 cm and the ePTFE tubing 252 has a length of about 0.5 cm to about 20 cm per end. In preferred embodiments, the silicone section 278 has a net length of about 7 cm to about 30 cm and the ePTFE tubing 252 has a length of about 1 cm to about 10 cm per end, and in most preferred embodiments, the silicone section 278 has a net length of about 10 cm to about 20 cm and the ePTFE tubing 252 has a length of about 3 cm to about 7 cm per end.

Another specific embodiment of the invention comprises a three-layer self-sealing device with inner and outer ePTFE graft material layers and a middle layer of everted silicone tubing. A three-layer device reduces the exposure of the self-sealing material to the vasculature and the body. This may improve the overall biocompatibility of the device compared to a two-layer design that exposes the self-sealing material to the vasculature. A three-layer device may be formed by sliding everted silicone tubing over a ePTFE graft material and bonding the two components. A larger diameter ePTFE graft is then slid over the everted silicone tube portion and bonded. Alternatively, the inner ePTFE layer may be bonded to the outer surface of non-everted silicone tubing and then everted with the silicone tubing. Like the two-layer design, the self-sealing middle layer is preferably shorter in length than both the inner and outer ePTFE layers to provide one or more ends without any silicone tubing. The inner and outer ePTFE layers may have the same or different lengths. Preferably, the outer ePTFE layer will have a shorter length than the inner ePTFE layer so that the ends of the device have a thickness comparable to traditional grafts. While an inner layer that is shorter than the outer layer will achieve a similar end thickness, such a configuration places the transition between the two components on the inner lumen of the device, rather than the outer surface, which may exhibit more turbulent flow and therefore have reduced hemocompatibility.

In a three-layer design, the ID of the everted silicone tubing is typically larger than the ID of the inner ePTFE graft, and the ID of the outer ePTFE component is larger than the everted silicone tubing. In one specific example, 7.5 mm ID silicone tubing is everted and slide over a 6 mm ePTFE graft. A larger, 8.5-9.5 mm ePTFE graft tubing is then slid over the everted silicone tube portion of the above assembly. In other embodiments of the invention, the inner graft material may have an ID of about 4 mm to about 10 mm, preferably about 5 mm to about 8 mm, and most preferably about 6 mm. The middle self-sealing material may have an ID of about 4 mm to about 11 mm and preferably about 5 mm to about 7 mm, and most preferably about 6 mm. The outer graft material may have an ID of about 5 mm to about 12 mm, preferably about 6 mm to about 10 mm, and most preferably about 7 mm to about 8 mm. The silicone tubing may have a length of about 5 to about 80 cm, preferably about 8 to about 25 cm, and most preferably about 10 to about 17 cm. The inner graft material may have a length of about 10 to about 100 cm, preferably about 20 to about 50 cm, and most preferably about 30 to about 40 cm, while the outer graft material may have a length of about 5 to about 82 cm, preferably about 8 to about 27 cm, and most preferably about 31 to about 42 cm.

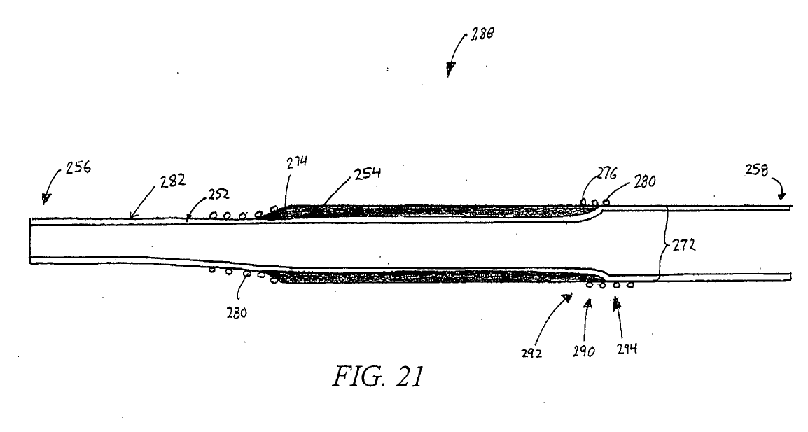

In still another embodiment of the invention, a tapered vascular access graft 288 is provided whereby one end 258 of the ePTFE tubing 252 of the graft 288 is larger than the other end 256 of the tubing 252. The difference in size of the ends 256, 258 may facilitate anastomosis of the graft 286 to an artery and a vein by providing a smaller ePTFE end 256 to attach to the smaller artery and providing a larger ePTFE end 258 to attach to the larger vein. The transition zone 290 between the smaller end 256 and the larger end 258 where the ID 270 changes may occur over the entire length of the ePTFE tubing 252 or one or more smaller segments of the ePTFE tubing 252. Thus, the transition of the ID 270 may be gradual or abrupt. The graft 288 may also be tapered at one or both ends 256, 258, e.g. about a 4 mm to about a 6 mm taper to keep the diameter at one or both anastomotic ends 256, 258 small while providing a larger diameter between the two anastomotic ends 256, 258 to facilitate needle insertion. In some embodiments, as shown in FIG. 21, the instant-access material 254 may be located on the exterior surface 282 of the tubing 252 about the smaller diameter portion 292 of the transition zone 290 and optionally extending onto the tubing 252. This location may be advantageous because it may ameliorate an increase in the OD 272 of the graft 288 that would have occurred had the instant-access material 254 been located along a section of the tubing 252 with a larger diameter 272. The ends 274, 276 of the instant-access material 254 are also preferably tapered to provide a smoother transition between the exterior surface 282 ePTFE tubing 252 and the ends 274, 276 of the instant-access material 254. As with other embodiments, anti-kink structures 280 may be provided about either or both ends 274, 276 of the instant access material 254. In an alternative embodiment, the instant-access material 254 may be located within the lumen 268 of a tapered graft 288 at about the larger diameter section 294 of the transition zone 290. The ends 274, 276 of the instant-access material 254 are also preferably tapered to provide a smoother transition of the graft lumen 268 between the ePTFE tubing 252 and the ends 274, 276 of the instant-access material 254.

The various instant-access graft embodiments above may be provided in different lengths to accommodate different tunnel length required by heterogeneous patient populations. The graft embodiments may also be provided with one or more separate sections connectable by graft section connector, which are either trimmable or adjustable to the desired length, or have selectable section lengths that can be combined to the desired length.

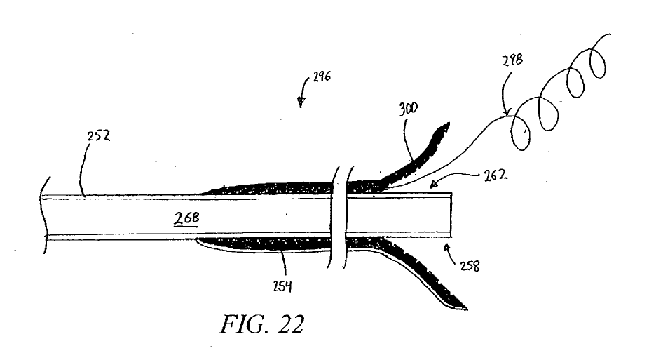

An adjustable length graft may be provided by a graft section that can delaminate the instant access material for removal to form an ePTFE-only end section of the graft. Referring to FIG. 22, one embodiment of an adjustable length graft 296 comprises an unwinding member 298 embedded within the instant access material 254 or between the instant access material 254 and the ePTFE tubing 252. After the graft 296 is cut or trimmed to its desired length, the unwinding member 298 may be peeled away, similar to a peel-away catheter, causing a portion 300 of the instant access material 254 to separate from the ePTFE tubing 252. The instant access material 254 may then be removed without removal of the underlying ePTFE tubing 252. This allows, for example, a surgeon to first trim one end 258 of the graft 296 to a desired length and then remove a segment 300 of instant-access material 254 to create an ePTFE-only end section 262 to facilitate anastomosis. In one embodiment, the instant access material 254 comprises silicone and a helical winding 298 embedded within the silicone 254 proximate to the ePTFE tubint 252 such that upon pulling of the helical winding 298, the silicone 254 delaminates from the ePTFE 252. The delaminated portion 300 of silicone 254 and unwound winding 298 would subsequently be trimmed off. Alternatively the instant access material 254 and winding member 298 may be in the lumen 268 of the ePTFE graft 296 and delaminate from the lumen 268.

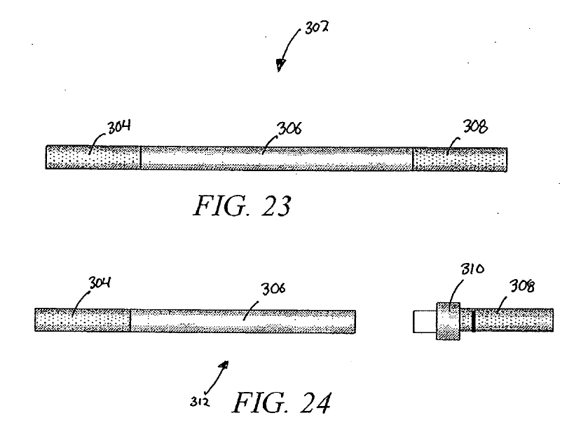

FIGS. 23 and 24 depict embodiments of an instant-access vascular graft 302, 312 comprising a first ePTFE-only end section 304 configured for arterial anastomosis, an instant-access material section 306, a second ePTFE-only end section 308 configured for venous anastomosis. The three sections 304, 306, 308 may be integrally formed during manufacture, or may use one or more graft section connectors 310 for joining two or more of the sections 304, 306, 308, as illustrated in FIG. 24. The number of graft section connectors 310 provided depends upon whether the instant-access material section 306 is integrally formed with either ePTFE-only end section 304, 308, if at all. An instant-access graft 302 comprising two or more segments joined by graft section connectors 310 allows, but does not require, each ePTFE end 304, 308 to be anastomosed to a blood vessel separately and then joined with the other sections 304, 306, 308 afterwards. It may be easier for a surgeon to anastomose one end of the graft 304, 308 without the bulk of the instant access section 306 with or without the other end 304, 308 of the graft 302 dangling during the anastomosis procedure. In other embodiments, however, both ends 304, 308 of the graft 302 may be joined by the connector(s) 310 before anastomosis is initiated. A multi-segment graft 312, may also allow one or more segments 306 of the graft 312 to be trimmed to the desired length before being joined by the connector(s) 310. This embodiment of the graft 312 preserves the benefits of an instant-access graft with ePTFE-only ends 304, 308 that a surgeon is familiar with while providing a graft 312 with an instant-access segment 306 that can be trimmed and tailored to the particular patient. This not only optimizes the length of the graft 312 for a particular patient, but may also reduce the stocking requirements for the hospital or surgical center by eliminating the need to stock multiple sizes of non-trimmable fixed-length grafts 302. In the preferred embodiment depicted in FIG. 24, the instant-access section 306 is integrally formed with one of the ePTFE sections 304 and therefore only requires one graft section connector 310 to join the three sections 304, 306, 308 together. Embodiments having only one graft section connector 310 may reduce the risk of accidental separation of the graft 312 by eliminating one site of potential disconnection.

Alternatively, each section 304, 306, 308 of the graft 312 may be packaged separately or together with multiple sizes which can be mixed and matched to provide the desired graft length or other graft characteristics. By packaging each component separately, however, waste of any one component may be reduced.

In further embodiment of the invention, a two-section device may be provided with a first section having a tapered anastomotic element that is integrally attached to a connector having about a 6 mm or 7 mm ID, and a second section configured with a final ID of about 4 mm and configured for arterial anastomosis. The differences in the internal diameters of the two sections gradually taper in order to reduce turbulence.

2. Temporary Access of the Vascular Access System

a. Temporary (pull out or tear-away) catheter

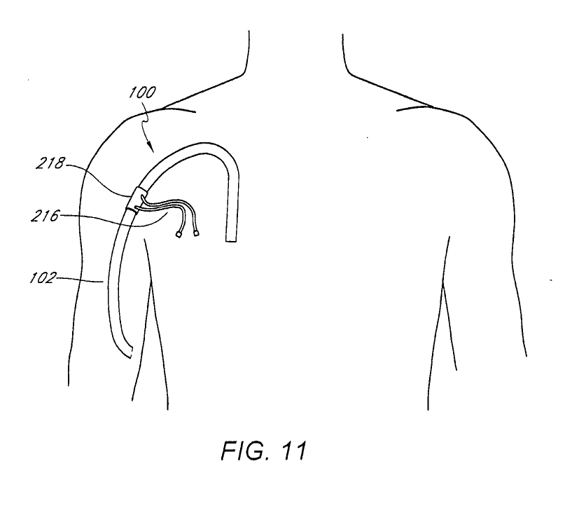

“Temporary” refers to a catheter being used short-term (about 90 days or less, but typically about a month or less) and configured to facilitate abandonment or removal after that time. Such a device could be used in the same manner as current hemodialysis catheters except it is expected to be abandoned or removed after limited use. A temporary catheter may be connected or formed with the permanent portion of the VAS so that both can be implanted in a single procedure, but later separated or severed when no longer needed. In some embodiments, as shown in FIG. 11, the temporary catheter 216 protrudes from the skin to eliminate the need to pierce the skin during use. Thus, one advantage of a temporary catheter 216 is that it would allow dialysis to be performed immediately after surgical implantation of the VAS 100 without the severe pain associated with needle sticks immediately following surgery (as is experienced with current instant stick grafts). Another possible advantage of abandoning or removing the catheter after a limited time period is that it will decrease the likelihood of infection, especially risks associated with long-term use of hemodialysis catheters and/or with vascular access extending from out of the skin. More than one temporary catheter may be provided.

In one embodiment, the temporary catheter 216 comprises a conduit with at least one lumen, but preferably at least two lumens, which are attached to the connector 218 of the VAS 100. In other embodiments, the temporary catheter may be attached at other locations of the VAS 100. With a single lumen, infusions or blood draws may be performed from the temporary catheter device, but dialysis is more difficult to perform due to recirculation. With two or more lumens, dialysis may be performed through the temporary catheter while the graft section 102 of the VAS 100 is healing-in (typically less than about one month). Once the graft section 102 is healed-in and the patient is able to dialyze through their VAS 100, the temporary catheter 216 is disabled by removing at least a portion of the temporary catheter device 216. It is desirable to disable the temporary catheter 216 because catheters which exit the skin have a higher long-term infection rate when compared to subcutaneous grafts. The temporary catheter may optionally have a Dacron cuff near the exit site in order to reduce the rate of infection.

### i. Seal Using Compressive Material at Junction

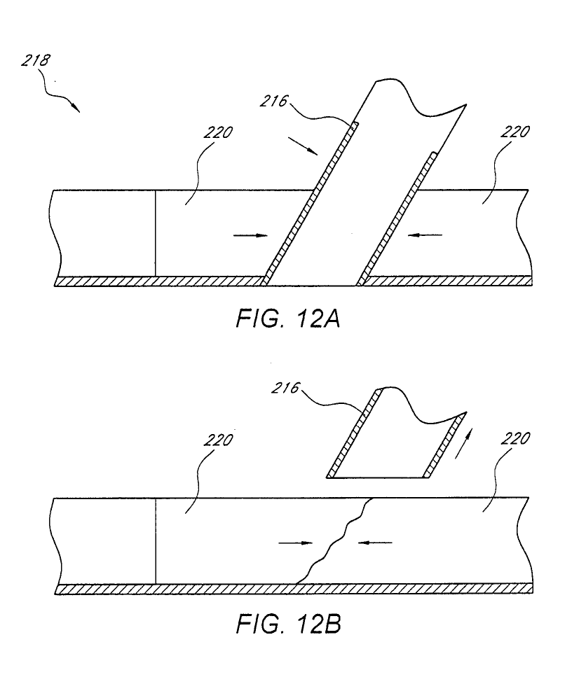

Referring to FIGS. 12A and 12B, in one embodiment of the invention, a compressive material 220 is incorporated into the conduit connector 218 and the temporary catheter 216 is attached to the connector 218 at the point of manufacture. The temporary catheter is used for about 90 days or less, but preferably less than about 1 month, and after that time, is removed in a manner similar to removing current hemodialysis catheters—it is pulled out from the site where the catheter exits through the skin. When the catheter 216 is pulled from the connector site, the compressed material 220 in the connector 218 seals the hole where the catheter 216 was removed, as shown in FIG. 12B.

### ii. Seal Using Flap at Junction

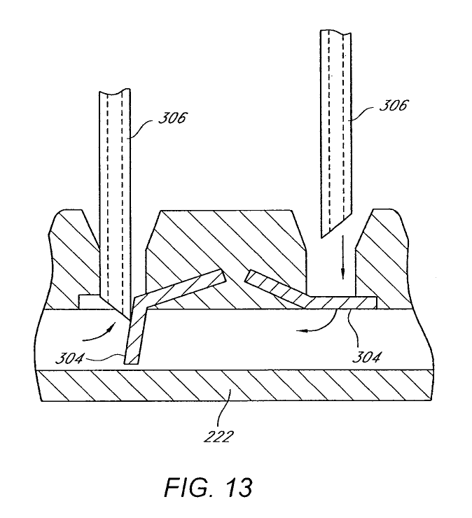

Alternatively, instead of employing a compressive material to seal off the hole in the connector when the temporary catheter is removed, a biased flap of material, similar to the needle access check valve as depicted in FIG. 13, may be adapted to provide a opening to the blood passageway when engaged to a temporary catheter or other access device. Upon removal of the temporary catheter, the biased flap resumes its bias so that the flap can cover or seal the hole.

### iii. Mechanical Valve at Junction

Another alternative embodiment comprises a mechanical valve instead of a flap to seal the hole in the connector when the temporary catheter is removed. One particular example is constructed using a self-closing valve set in the conduit connector or other section of the VAS. The temporary catheter fits into and may inhibit the self-sealing connection feature until removal.

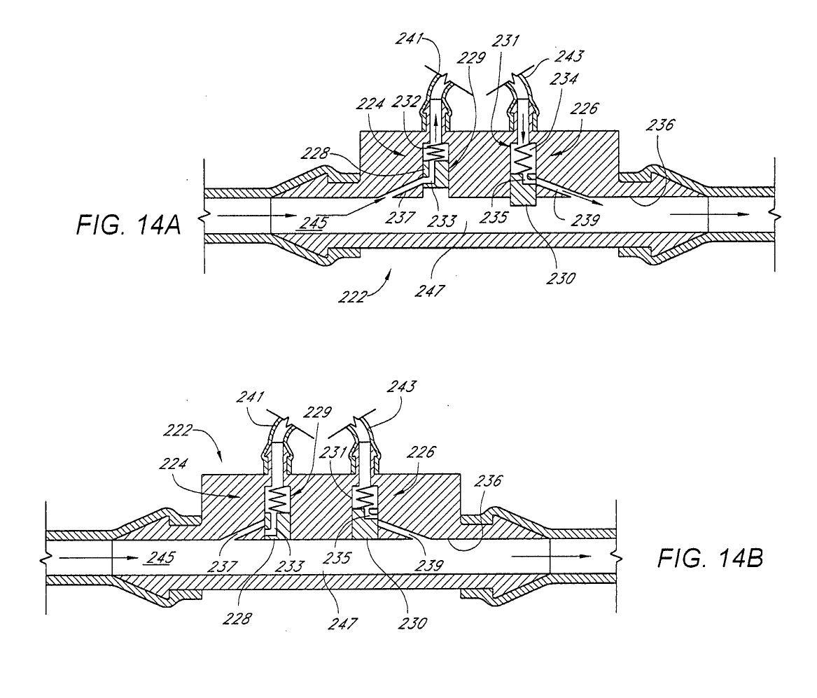

Referring to FIGS. 14A and 14B, the central hub of a connector 222 may be used to house a set of mechanical valves 224, 226. One valve is the outlet 224 while the other is the inlet 226. This embodiment involves creating a pressure differential to move pistons 228, 230 along internal pathways 229, 231 between an open position and closed position, as shown in FIGS. 14A and 14B, respectively. These pistons 228, 230 may be connected to springs 232, 234 for equilibrium positioning. In the resting or closed position depicted in FIG. 14B, the piston heads 228, 230 would be flush with the inside surface 236 of said connector 222 and the piston conduits 233, 235 are out of alignment with inlet and outlet conduits 237, 239. As pressure and/or vacuum is applied from the connected tubing 241, 243, the pistons 228, 230 move from resting position to the open position to align the piston conduits 233, 235 with the inlet and outlet conduits 237, 239 so that may flow commence. When the pressure and/or vacuum is shut off, the pistons 228, 230 return to resting position, inhibiting any flow. In some further embodiments, one or both of the pistons may be configured to protrude into the connector's lumen 245 in order to reduce or eliminate the flow through the middle portion 247 of the connector 222. This may be desirable because it will help prevent or eliminate recirculation of the blood during dialysis (i.e. prevents blood from flowing directly from the outlet port from the temporary catheter and then into the inlet port of the temporary catheter).

### iv. Seal With Insert Plus With Positive Locking Stop

In another alternative embodiment, the temporary catheter may be completely separated from the connector. A plug is inserted through the temporary catheter and locks into place in order to seal the hole(s) in the connector.

b. Abandoned Catheter Section

### i. Seal Through Lumen Using Plug/Mandrel With Positive Locking Stop

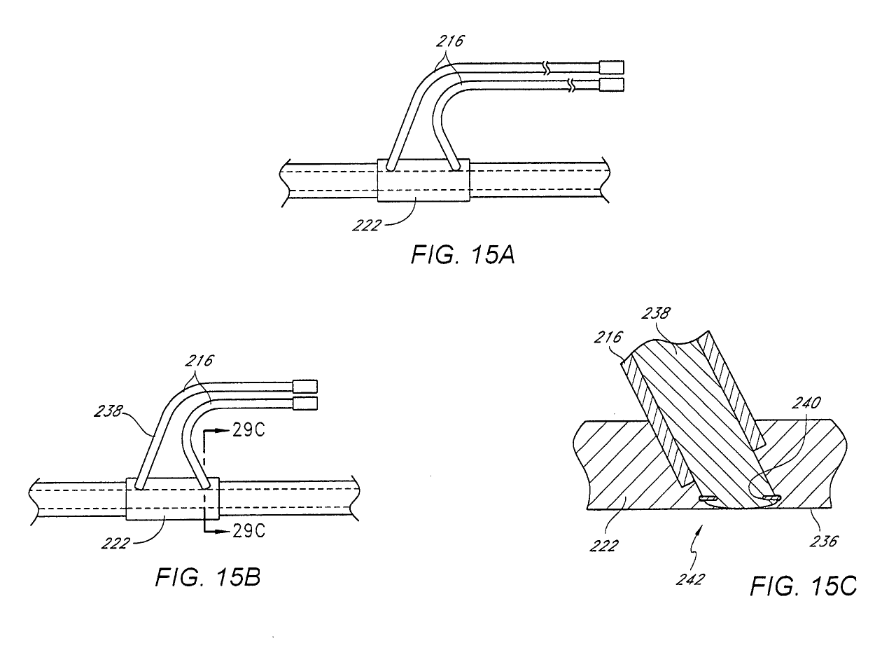

Referring to FIGS. 15A to 15C, in one embodiment, a plug 238 is inserted through the temporary catheter 216 and locked into place in order to seal the hole in the connector 222. The plug 238 may be configured such that it is generally flush with the lumen 236 of the connector 222, or where the plug minimizes sharp edges, bumps, holes or other surface irregularities that would cause turbulence as this could lead to thrombus buildup and eventual device occlusion. In this embodiment, the subcutaneous portion of the temporary catheter 216 remains in place and therefore a portion of the plug 238 may stay in the catheter 216. In some embodiments, as shown in FIG. 15C, one or more complementary detents/protrusions 240, 242 may be provided to further control the relative position of the plug 238 with the lumenal surface 236 of the connector 222.

### ii. Inject Sealing Compound into Lumen

In one embodiment of the invention, a material that has the ability to solidify may be used to plug the lumens. There are several materials that may be used, such as cements, epoxies, and polymers. A preferred material is Onyx® from Micro Therapeutics, Inc. Onyx® is a liquid embolization material that may be injected through the lumens under fluoroscopic or other type of visualization. When the material comes in contact with the flowing blood, it will form a smooth surface and become solid through a precipitation reaction (e.g. DMSO is exchanged with the water in blood). More specifically, Onyx® is a liquid mixture of ethylene vinyl alcohol co-polymer (EVOH) dissolved in dimethyl sulfoxide (DMSO). Micronized tantalum powder is suspended in the liquid polymer/DMSO mixture to provide fluoroscopic visualization. The Onyx material is delivered in a liquid phase to fill the catheter lumens under fluoroscopic control. Upon contact with blood (or body fluids) the solvent (DMSO) rapidly diffuses away, causing in-situ precipitation of a soft radiopaque polymeric material. After the lumen is filled and the filling material has solidified, the temporary catheter may be cut so it lies subcutaneously. (Clinical Review of MTI, Onyx® Liquid Embolization System, available at http://www.fda.gov/ohrms/dockets/ac/03/briefing/3975b1-02-clinical-review.pdf, accessed Aug. 29, 2005).

### iii. Plug Lumen at Proximal End Only

In another embodiment, the proximal end of the temporary catheter 216 is sealed using a plug, clamp, winding, suture or other method and the temporary catheter 216 is cut subcutaneously. The temporary catheter 216 may be sealed then cut, or cut then sealed. The disadvantage of this method is that there is a chance of producing turbulence where the temporary catheter ends inside the connector because there would be an abrupt transition and a blind end where blood stasis will occur.

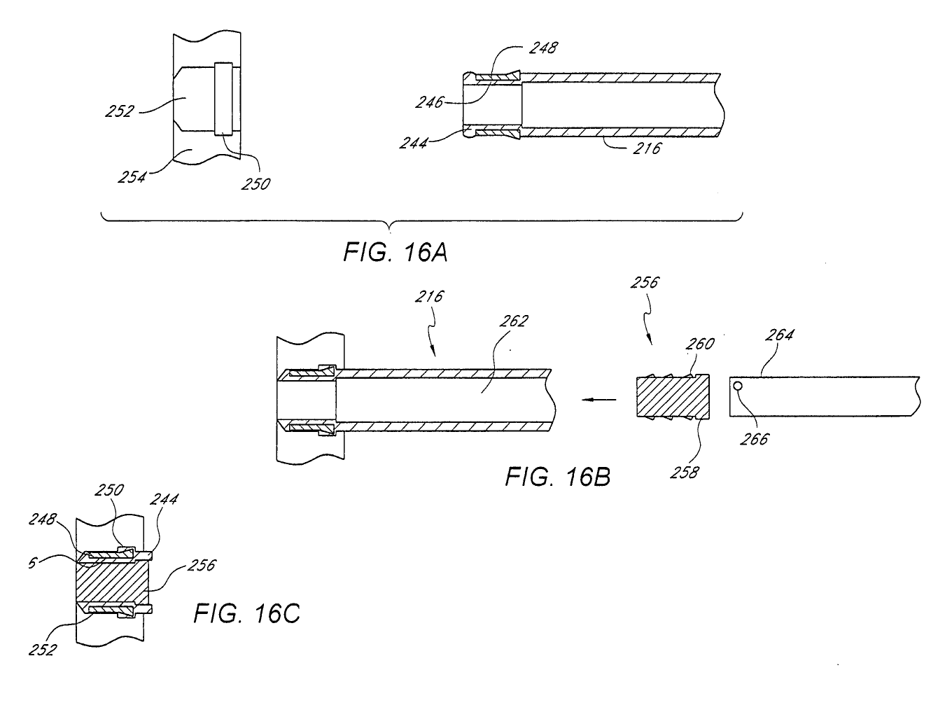

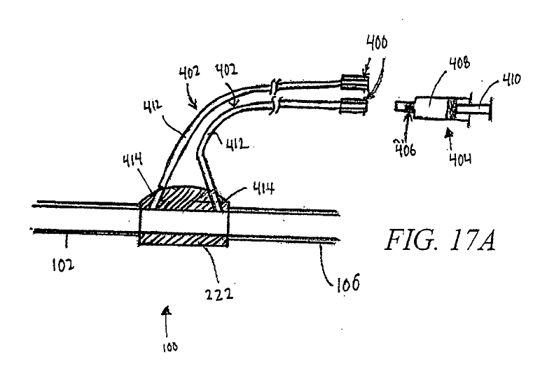

In particular one embodiment, depicted in FIG. 16A, the temporary catheter 216 and connector 2 form a complementary lock/latch mechanism, whereby the end 244 of the temporary catheter 216 comprises a hard material, either metal or plastic, and a recess 246 containing a biased-split ring 248, and is capable of interfacing with a coupling lumen 252 in the wall 254 of the conduit connector. As shown in FIG. 16B, the coupling lumen 252 is configured with a complementary groove 250 whereby when the temporary catheter 216 is fully inserted into the coupling lumen 252, the biased-split ring 248 can snap into the groove 250 to lock the temporary catheter 216 into the coupling lumen 252 on the conduit connector. In an alternative embodiment, the recess and biased-spit ring may be positioned in the coupling lumen while the end 244 of the temporary catheter 216 has a complementary groove. One of skill in the art will understand that any of a variety of other securing structures may also be used, including but not limited to biased projecting prongs and threaded rotation interfaces.

Once the temporary catheter 216 is no longer needed, the temporary catheter 216 may be plugged or filled, and severed about its proximal end 244. By severing the temporary catheter 216, the amount of foreign body remaining in the patient is reduced, which in turn may reduce the risk of infection, immune system response, and/or cosmetic effect.

Referring back to FIG. 16B, a plug 256 with an insertion stop 258 and one or more ramped edges 260 along its surface is inserted into the lumen 262 of the temporary catheter 216. The ramped edges 260 of the plug 256 provide resistance to backout for the plug 256 while the insertion stop 258 allows the plug 256 to seat in the end 244 of the temporary catheter 216 without protruding excessively past the wall 254 of the connector. The plug 256 is inserted into the temporary catheter 216 using a catheter cutter 264 with a retractable blade 266. The catheter cutter 264 is used to push the plug 256 into the catheter lumen 262. Once the plug 256 is in place, the retractable blade 266 is extended from the catheter cutter 264 and the catheter cutter 264 is rotated or otherwise manipulated to sever at least a portion of the temporary catheter 216 from its end 244. The retractable blade 266 is retracted and the separated portion of the temporary catheter 216 is removed from the patient along with the catheter cutter 264. The end 244 of the temporary catheter 216 and plug 256 remain in the coupling lumen 252 of the wall 254 of the connector and seal it from blood leakage.

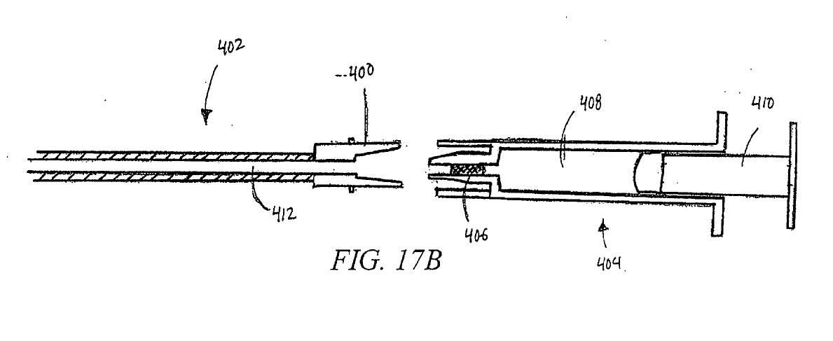

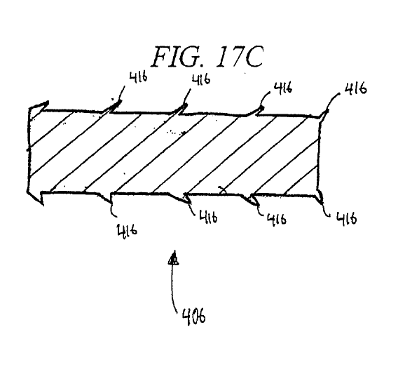

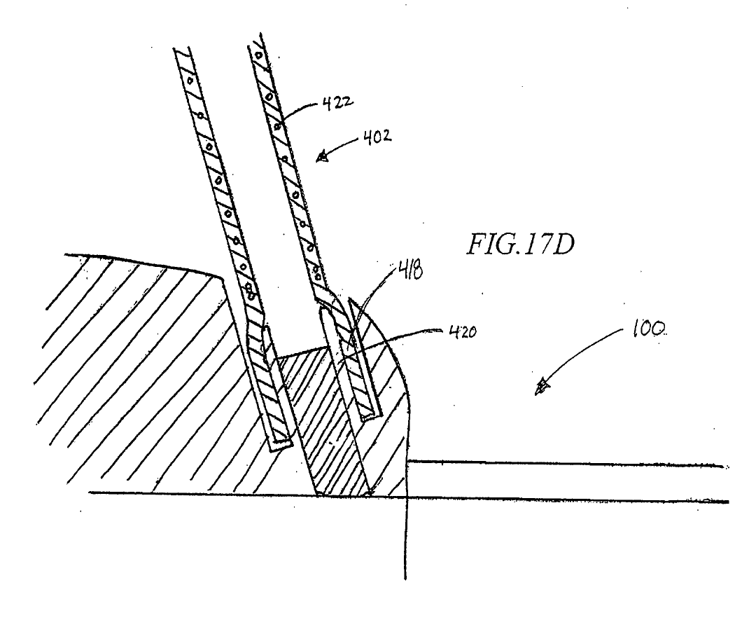

In one specific embodiment depicted in FIGS. 17A and 17B, the exposed ends 400 of the temporary or auxiliary catheters 402 are provided with connector configurations to allow engagement of a syringe 404. The syringe 404 contains a plug 406 of material and a delivery fluid 408 such that when the syringe 404 is attached and the plunger 410 of the syringe 404 is actuated, the delivery fluid 408 will propel the plug 406 through the lumen 412 of the auxiliary catheter 402 and firmly lodge and seal off the distal end 414 of the auxiliary catheter lumen 412 from the other portions of the VAS 100. Preferably, the syringe 404 and auxiliary catheter 402 are configured so only a small volume of delivery fluid is needed to implant the plug 406 and release the auxiliary catheter 402. In a preferred embodiment, a 1.5 cc syringe may be used, wherein about 1 cc of delivery fluid 408 is used to delivery the plug 406 and about 0.5 cc of delivery fluid 408 is used to pressurize and release the auxiliary catheter 402. FIG. 17C depicts one embodiment of the plug 406. The plug has an elongate shape with a cross-sectional shape complementary to the cross sectional shape of the auxiliary catheter lumen, which is typically circular. The outer surface of the plug has one or more circumferential flexible projections or flaps 416. The one or more flaps 416 create a seal with the lumen 412 of the auxiliary catheter 402, thus providing the ability to propel the plug 406 by hydraulic pressure. The flexibility of the flaps 416 allow the maintenance of a seal with the catheter lumen 412 despite variations in the auxiliary catheter lumen size or surface, and also reduce the frictional resistance between the plug 406 and the auxiliary catheter lumen 412, which may reduce the magnitude of pressure required to propel the plug 406. Typically, the flaps 416 on the plug 406 are angled to facilitate movement in one direction within the lumen 412 while resisting motion in the opposite direction within the lumen 412. The angulation may also improve the sealing properties of the plug 406. In some embodiments of the invention, proper positioning of the plug 406 in the distal portion 414 of the auxiliary catheter lumen 412 may be facilitated by complementary grooves or ridges located on the lumenal surface of the auxiliary catheter at the desired plug position. A taper fit or shoulder between the plug and the distal end of the catheter is preferred, but not required, to restrict the plug from going to far and to achieve a tight seal.

Once the plug 406 is in place, the attached syringe 404 is able to generate increased hydraulic pressure within the proximal lumen 412 of the auxiliary catheter 402, due to the fluid seal formed by the plug 406 at the distal lumen end 414 of the auxiliary catheter 402. The ability to increase the hydraulic pressure may be used to at least partially separate, loosen or unlock the auxiliary catheter 402 from the remaining portions of the VAS 100. Referring to FIG. 17D, the distal end 418 of the auxiliary catheter 402 may be an elastic female connector end configured to engage a male connector end 420 on the VAS 100 and to form a sealed connection. In other embodiments, the male/female connector locations may be reversed. The elastic property of the distal female end 418 of the auxiliary catheter 402 may be due to the elastic material and/or an elastic reinforcement element, such as a winding. Elasticity in other portions of the auxiliary catheter 402, if undesirable, may be reduced by reinforcement of the elastic wall with metallic or nylon windings 422 as discussed elsewhere herein. When the hydraulic pressure is sufficiently increased by the syringe 404, the elastic connection between the female and male connector ends 418, 420 is loosened and the auxiliary catheter 402 may be separated from the rest of the VAS 100. Some fluid may leak into the subcutaneous tissue when the connectors are loosened. In some instances, the available fluid in the syringe is leaked into the tissue before the auxiliary catheter has completely separated. When this occurs, the same or different syringe will additional fluid may be used to complete the separation procedure. Preferably, use of excess amounts of fluid to separate the auxiliary catheter should be avoid given the inability or reduced ability of renal failure patients to rid of excess fluid. In other embodiments of the invention, pressurization of the auxiliary catheter lumen 412 only partially loosens the connection of the auxiliary catheter 402 sufficiently to reduce the force required to separate the auxiliary catheter 402 from the remaining portions of the VAS 100 but not enough to break the fluid seal between the connector ends 418, 420. This may prevent leakage of syringe fluid into the interstitial space.

In an alternate embodiment, the distal end of the auxiliary catheter may be non-elastic or may be elastic but undergo plastic deformation at particular hydraulic pressures. The auxiliary catheter is configured to deform for a substantial period of time or permanently unlock when the pressure within the auxiliary catheter exceeds a set pressure level, thereby providing a longer window for disconnecting the auxiliary catheter. In other embodiment, the distal end of the auxiliary catheter may be constructed using a different formulation of the same base material as the rest of the auxiliary catheter, but with a different durometer. The distal end may be formed simultaneously as part of the entire auxiliary catheter or may be made separately and later bonded to the other section of the auxiliary catheter.

c. Implantation of temporary access

In one embodiment for implanting the VAS with a temporary access structure, the pathway for the catheter section of the VAS is tunneled first, the pathway for the pre-connected graft section of the VAS is tunneled next, followed preferably by the tunneling of a pathway from the intermediate access site to a temporary catheter exit site. It is preferable that the temporary catheter be located at a tunneled exit site rather than project directly out of the intermediate access site where the catheter section is attached to the graft section, in order to reduce the risk of infection of the main VAS assembly. By increasing the distance between the connector to the skin site where the temporary catheter exits the body, infection of the connector is reduced. After the temporary catheter is tunneled from the chest to the connector, the catheter is locked or latched into the connector, as described in embodiments disclosed above. The temporary catheter may also be tunneled from the connector to the exit site.

While this invention has been particularly shown and described with references to embodiments thereof, it will be understood by those skilled in the art that the various changes in form and details may be made therein without departing from the scope of the invention. For all of the embodiments described above, the steps of the methods need not be performed sequentially. Furthermore, any references above to either orientation or direction are intended only for the convenience of description and are not intended to limit the scope of the invention to any particular orientation or direction.

1-98. (canceled)

99. A biocompatible vascular graft, comprising a tubular leak-resistant material having an outer surface, an inner surface, a first end, a second end, a longitudinal axis, and an inner lumen between the first end and the second end, wherein at least a portion of the tubular leak-resistant material is circumferentially compressed.

100. The biocompatible graft as in claim 99, wherein the tubular leak-resistant material has an everted configuration.

101. A biocompatible graft as in claim 100, further comprising a stretch-resistant layer bonded to a leak-resistant material, wherein the stretch-resistant layer resists expansion of the leak-resistant material that would substantially result in opening and leakage of any needle puncture sites in the leak-resistant material.

102. The biocompatible graft as in claim 101, wherein the leak-resistant material comprises a silicone layer and the stretch-resistant layer comprises an ePTFE layer.

103. The biocompatible graft as in claim 101, wherein the leak-resistant material comprises a leak-resistant tubing material and the stretch-resistant layer comprises stretch-resistant tubing material.

104. The biocompatible graft as in claim 102, wherein the ePTFE layer is ePTFE tubing comprising a length, an exterior surface, an outer diameter, a first end, a second end, a lumen therebetween, and a inner diameter.

105. The biocompatible graft as in claim 104, wherein the silicone layer comprises silicone tubing having a first end and a second end.

106. The biocompatible graft as in claim 105, wherein the silicone tubing is applied to the exterior surface of the ePTFE tubing.

107. The biocompatible graft as in claim 105, wherein the silicone tubing is applied to the lumen of the ePTFE tubing.

108. The biocompatible graft as in claim 105, wherein the silicone tubing has a length less than the length of the ePTFE tubing.

109. The biocompatible graft as in claim 106, further comprising a layer of ePTFE overlayed on the silicone tubing.

110. The biocompatible graft as in claim 109, wherein the overlayed layer of ePTFE completely covers the silicone tubing.

111. The biocompatible graft as in claim 108, wherein the silicone tubing is located at least about 0.25 cm from the first end of the ePTFE tubing.

112. The biocompatible graft as in claim 111, wherein the silicone tubing is located at least about 0.5 cm from the first end of the ePTFE tubing.

113. The biocompatible graft as in claim 111, wherein the silicone tubing is located at least about 0.25 cm from the second end of the ePTFE tubing.

114. The biocompatible graft as in claim 112, wherein the silicone tubing is located at least about 1 cm from the first end of the ePTFE tubing.

115. The biocompatible graft as in claim 112, wherein the silicone tubing is located at least about 0.5 cm from the second end of the ePTFE tubing.

116. The biocompatible graft as in claim 114, wherein the silicone tubing is located at least about 1 cm from the second end of the ePTFE tubing.

117. The biocompatible graft as in claim 105, wherein the lumen of the ePTFE tubing comprises a luminal smaller diameter zone, a luminal transition zone and a luminal larger diameter zone.

118. The biocompatible graft as in claim 117, wherein the silicone tubing is applied to the lumen of the ePTFE tubing about the luminal transition zone and the luminal larger diameter zone.

119. The biocompatible graft as in claim 105, wherein the exterior surface of the ePTFE tubing comprises an exterior smaller diameter zone, an exterior transition zone and an exterior larger diameter zone.

120. The biocompatible graft as in claim 119, wherein the silicone tubing is applied at least to the exterior surface of the ePTFE tubing about the luminal transition zone and the luminal smaller diameter zone.

121. The biocompatible graft as in claim 105, wherein the silicone tubing is applied to the exterior surface of the ePTFE tubing.

122. The biocompatible graft as in claim 102, wherein the leak-resistant material and stretch-resistant layer form an instant access segment located between a first ePTFE end segment and a second ePTFE end segment.

123. The biocompatible graft as in claim 122, wherein the first ePTFE end segment and instant access segment are integrally formed.

124. The biocompatible graft as in claim 122, wherein the first ePTFE end segment and instant access segment are joined by a segment connector.

125. The biocompatible graft as in claim 108, further comprising at least one anti-kink structure about the first end of the silicone tubing or the second end of the silicone tubing.

126. The biocompatible graft as in claim 125, further comprising anti-kink structures about both the first end of the silicone tubing and the second end of the silicone tubing.

127. The biocompatible graft as in claim 106, further comprising a separation member embedded generally within the silicone tubing, or between the silicone tubing and the ePTFE tubing.

128. The biocompatible graft as in claim 127, wherein the separation member is a helical unwinding member.

129. The biocompatible graft as in claim 101, wherein the leak-resistant material is longitudinally compressed.

130. The biocompatible graft as in claim 99, wherein the circumferential compression of the leak-resistant material is inherent in the configuration of the leak-resistant material.

131. The biocompatible graft as in claim 99, wherein the outer surface of the tubular leak-resistant material about has a circumferential tension that radially compresses the tubular leak-resistant material about the inner surface of the tubular leak-resistant material.

132. The biocompatible graft as in claim 99, wherein the tubular leak-resistant material exhibits increasing compression from its outer surface to its inner surface.

133. The biocompatible graft as in claim 99, wherein the outer surface of the tubular leak-resistant material is in an expanded configuration and the inner surface of the tubular leak-resistant material is in a compressed configuration.

134. The biocompatible graft as in claim 99, further comprising a radial compression structure.
